(19) United States
(12) Patent Application Publication
(10) Pub. No.: US 2024/0390620 A1
(43) Pub. Date: Nov. 28, 2024

(54) MEDICAL SYSTEM USING ADVANCED ANALYTICS AND MACHINE LEARNING

(71) Applicant: StatOS, Inc., Los Angeles, CA (US)

(72) Inventors: Michael J. Horzewski, San Jose, CA (US); Gajus Worthington, Portola Valley, CA (US); Ryan Van Wert, San Francisco, CA (US); David B. Camarillo, Stanford, CA (US)

(21) Appl. No.: 18/792,945

(22) Filed: Aug. 2, 2024

Related U.S. Application Data
(63) Continuation of application No. 18/532,991, filed on Dec. 7, 2023.
Application No. 18/532,991 is a continuation of application No. PCT/US2022/032481, filed on Jun. 7, 2022.
(60) Provisional application No. 63/208,439, filed on Jun. 8, 2021.

(51) Int. Cl. A61M 16/00; G16H 10/60; G16H 20/40

(52) U.S. Cl. CPC A61M 16/026; G16H 10/60; G16H 20/40; A61M 2205/3303; A61M 2205/502; A61M 2230/40

(57) ABSTRACT

Described herein are devices and methods for determining or administering a therapy to a patient. Devices and methods described herein are configured in different embodiments to carry out different types of treatments in different healthcare settings. In some embodiments, the devices and methods described herein use advanced analytics to administer a therapy to a patient.

### CROSS-REFERENCE

This application is a continuation of International Application No. PCT/US2022/032481, filed Jun. 7, 2022, which claims the benefit of U.S. Provisional Application No. 63/208,439, filed Jun. 8, 2021, the contents of which are incorporated by reference herein in their entirety.

### BACKGROUND OF THE INVENTION

Patients may be unable to independently support their cardiovascular system. Such patients may be placed on a ventilator to supplement use of patient's own lungs. These ventilators may be difficult to program or operate.

### SUMMARY OF THE INVENTION

Described herein are devices and methods for determining or administering a therapy to a patient. Devices and methods described herein are configured in different embodiments to carry out different types of treatments in different healthcare settings. In some embodiments, the devices and methods described herein use advanced analytics to administer a therapy to a patient.

An aspect of the present disclosure provides a system for providing respiratory support to a patient, the system comprising: (a) a ventilator; (b) a processor; and (c) a non-transitory computer-readable medium including software comprising artificial intelligence, the software configured to cause the processor to: (i) receive an input comprising a quantifiable parameter from a patient receiving pulmonary support from the ventilator; (ii) analyze the input using the artificial intelligence; and (iii) modify performance of the ventilator resulting in a quantifiable improvement in the measured parameter.

In some embodiments, the quantifiable parameter comprises ventilator data. In some embodiments, the ventilator data comprises tidal volume (VT), respiratory rate, FiO2, CO2, breathing gas flow rate, inspiration time, peak airway pressure, peak end-expiratory pressure (PEEP), or apnea. In some embodiments, the quantifiable parameter comprises ventilator waveform data. In some embodiments, the quantifiable parameter comprises patient data. In some embodiments, the patient data comprises arterial blood gas measurements (ABG), EKG signal, blood pressure, body temperature, heart rate, respiration rate, general health, or patient position. In some embodiments, the system further comprises audio/visual equipment. In some embodiments, the audio/visual equipment comprises a camera. In some embodiments, the input comprises an image from the camera. In some embodiments, the artificial intelligence comprises a machine learning algorithm. In some embodiments, the machine learning algorithm comprises a deep learning algorithm. In some embodiments, the software further causes the processor to predict a patient outcome. In some embodiments, the system further comprises a database. In some embodiments, the software further causes the processor to receive an input from the database. In some embodiments, input from the database comprises information about other patients similar to the patient with respect to demographic, disease, conditions, or treatments.

Another aspect of the present disclosure provides a system for providing respiratory support to a patient, comprising: (a) a database; (b) a processor; and (c) a non-transitory computer-readable medium including software comprising artificial intelligence, the software configured to cause the processor to: (i) receive, with the database, a plurality of quantifiable parameters from a plurality of patients who are receiving ventilator pulmonary support; (ii) analyze the plurality of inputs within the database using the artificial intelligence thereby generating an analysis result; and (iii) determine at least one ventilator parameter or patient parameter that when modified improves an outcome of a patient receiving ventilator pulmonary support.

In some embodiments, the plurality of quantifiable parameters comprises ventilator data. In some embodiments, the plurality of quantifiable parameters comprises one or more of tidal volume (VT), respiratory rate, FiO2, CO2, breathing gas flow rate, inspiration time, peak airway pressure, peak end-expiratory pressure (PEEP), or apnea. In some embodiments, the plurality of quantifiable parameters comprises ventilator waveform data. In some embodiments, the plurality of quantifiable parameters comprises patient data. In some embodiments, the patient data comprises one or more of arterial blood gas measurements (ABG), EKG signal, blood pressure, body temperature, heart rate, respiration rate, general health, or patient position. In some embodiments, the system further comprises audio/visual equipment. In some embodiments, the audio/visual equipment comprises a plurality of cameras. In some embodiments, the plurality of quantifiable parameters comprises images from the plurality of cameras. In some embodiments, the artificial intelligence comprises a machine learning algorithm. In some embodiments, the machine learning algorithm comprises a deep learning algorithm. In some embodiments, the software further causes the processor to predict potential patient outcomes. In some embodiments, the input from the database further comprises information about other patients similar to the patient with respect to demographic, disease, conditions, or treatments.

Another aspect of the present disclosure provides a system for providing respiratory support to a patient, comprising: (a) a ventilator; (b) a processor; and (c) a non-transitory computer-readable medium comprising software configured to direct the processor to: (i) receive an input from a user; (ii) configure a set of ventilation parameters from the input; (iii) initiate ventilation of the patient; (iv) monitor ventilation of the patient; (v) conduct a ready to wean trial upon the patient satisfying a ready to wean criteria; and (vi) alert the user when the ready to wean trial is successfully completed.

In some embodiments, the system further comprises a database. In some embodiments, the software further causes the processor to receive an input from the database. In some embodiments, the input from the database comprises information about other patients similar to the patient with respect to demographic, disease, conditions, or treatments. In some embodiments, the input from the database comprises information and guidelines from reference sources. In some embodiments, the input comprises arterial blood gas measurements (ABG), blood pressure, body temperature, heart rate, respiration, general health, or patient position. In some embodiments, the software causes the processor to prompt the user to adjust the set of ventilation parameters or to accept the set of ventilation parameters prior to initiating ventilation of the patient. In some embodiments, the software causes the processor, once the input from a user has been received, to output to the user an output. In some embodiments, the output comprises equipment settings, medications, ventilation parameters, patient position, diagnostic tests, or forecasting. In some embodiments, the system further comprises a handheld communication device. In some embodiments, the software causes the processor, once the input from a user has been received, to generate an output and send the output to the handheld communication device. In some embodiments, the software causes the processor to configure a set of baseline alarm parameters from the set of baseline ventilation parameters. In some embodiments, the software causes the processor to execute an algorithm comprising an alarm workflow when a parameter of the set of baseline alarm parameters is met. In some embodiments, the alarm workflow comprises modifying the set of ventilation parameters. In some embodiments, the ready to wean criteria comprise respiratory rate, tidal volume (VT), minute ventilation, PEEP, pH, PaO2/FiO2, pulmonary shunt fraction, negative inspiratory force, frequency, stable cardiovascular status, heart rate, hemoglobin, systolic blood pressure, temperature, or no or minimal vasopressor or inotrope. In some embodiments, the ready to wean trial comprises a spontaneous breathing trial (SBT). In some embodiments, the software causes the processor to determine a set of weaning parameters based on the ventilation parameters and prompt the user, if desired, to adjust the set of weaning parameters return to ventilating the patient according to the set of ventilation parameters, if the patient meets a set of failing criteria.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an embodiment of the O2U system.

FIG. 2 shows an embodiment of the control center.

FIG. 3A illustrates an embodiment of the display.

FIG. 3B shows an embodiment of the display comprising a large parameter display.

FIG. 3C shows an embodiment of the display comprising an audio/visual field.

FIG. 4A shows an embodiment of controlled equipment in perspective view.

FIG. 4B shows an embodiment of controlled equipment in side view.

FIG. 5 illustrates a schematic diagram of a data processing method for generating a machine learning model to optimize respiratory support for a patient.

FIG. 6 shows a method of tuning a predictive model's parameters based on patient input data resulting in a predictive model configured to optimize respiratory support for a patient.

FIG. 7 shows an operational flow method of implementing a machine learning model configured to optimize respiratory support for a patient.

FIG. 8 illustrates a computer system suitable for implementing the machine learning algorithm model described herein.

FIG. 9 shows an embodiment of an algorithm-based workflow.

FIG. 10 shows an embodiment of an algorithm-based alarm workflow.

### DETAILED DESCRIPTION OF THE INVENTION

### Introduction

Despite the necessity of mechanical ventilation in healthcare, complications such as infection or lung damage are possible if ventilation is performed improperly or for too long. Furthermore, the high level of training and skill required to operate certain pieces of medical equipment such as a ventilator can delay treatment reaching patients quickly or reaching patients who are geographically isolated, such as those in rural areas or combat zones. A related problem is the additional time expense incurred in situations where a healthcare provider is required to don personal protective equipment (PPE) to see a patient. According, the present invention seeks to overcome these limitations.

Additionally, rapid iterative processing with intelligent algorithms is extremely valuable in dynamic situations, such as where the patient's conditions are quickly changing, the treatment condition is rapidly changing, and/or the disease is evolving or just becoming understood. An example would be during the COVID-19 outbreak, ventilation strategies were varied throughout treatment centers with varying levels of success. Simultaneously, recent advancements in artificial intelligence and big data research have permitted insights to be gleaned from massive datasets that had previously resisted interpretation. Accordingly, the present invention further seeks to use advanced analytics to optimize the performance of medical equipment and methods of use thereof.

Such equipment and methods may provide several advantages. The information provided and/or generated by the equipment may reduce the knowledge base or skillset required to operate medical equipment. Such information may allow some physician tasks to be completed by non-physicians or personnel with less training than is required for typical equipment. Additionally, the equipment and methods may allow some specifically trained user tasks to be completed by a generalist. Such equipment and method may also expand the user base or useable market for complex medical equipment and methods. The equipment and methods may increase successful usage in rural areas and developing companies. The equipment and methods may permit the user to remain in a separate physical location from the patient while still providing healthcare. The equipment and methods may allow the user to see more patients in less time or to allow the same number of patients to be treated with less staff which may be particularly advantageous during crises such as a novel coronavirus disease outbreak (COVID-19). The equipment and methods may additionally find use in military settings where they may be used in the field by troops, combat medics, specialists, and physicians.

The equipment and methods may comprise technology from various medical specialties. The equipment and methods may comprise general medical equipment and methods of using such as cardiac monitors; IV pumps; blood gas analyzers; a ventilator and associated equipment; medical imaging equipment such as x-ray and ultrasound equipment; EKG interpretation; EEG interpretation and evaluation; intermittent pneumatic compression equipment; bed settings; and thermal (hot/cold) pads and blankets.

### Overview of the Device

The O2U System 100 can be used as part of the medical care during the treatment of one or more patients. The O2U System 100 may be used for treating a patient in an intensive care unit (ICU); however, the O2U System 100 and/or any of its components, individually or in combination, can be used in care of a patient, including but not limited to, care in the ICU, intensive therapy unit (ITU), critical care unit (CCU), emergency department (ED), regular units, outpatient centers, remote care, battlefield, and the like.

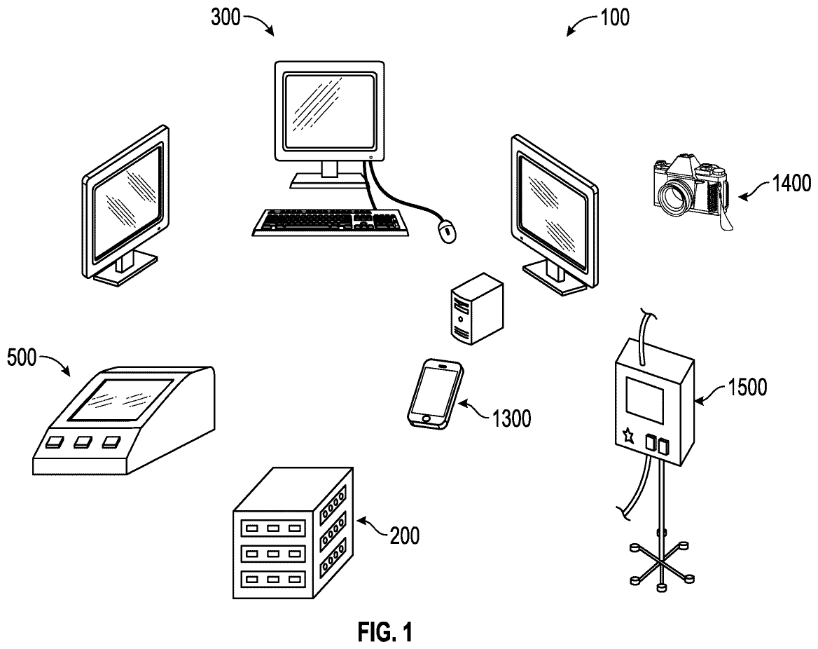

FIG. 1 illustrates an example of a O2U System 100, including one or more of, but not limited to, a Database 200; a Control Center 300; a Ventilator 500; a Handheld Communication Device 1300, such as a phone, tablet, portable computer; Audio/Visual Equipment 1400; and additional representative controlled equipment, such as an IV Pump 1500 that can be used as a system or individually in the care of a patient. It will be understood that though the O2U System 100, its components, and methods described herein are described primarily with reference to use in the ICU setting, they can also be used in any setting requiring medical equipment. Controlled equipment can be any component of the O2U System 100, such as the Control Center 300, Ventilator 500, Audio/Visual Equipment 1400, IV Pump 1500, it may also include such equipment as a blood gas analyzer, intermittent pneumatic compression (IPC), patient bed and bed settings (e.g. position), thermal (hot/cold) pads/blankets, and includes any Handheld Communication Device 1300 that is accessing or using one or more of the Database 200, O2U software, or communicating with other controlled equipment. The Database 200 may be contained within a piece of controlled equipment or be a separate entity.

### Apparatus for Data Input, Processing, and Storage

The Database 200 may receive, process, and/or store data from any controlled equipment, sensors or connected sensors (e.g. O2, CO2, heart rate, blood pressure, EKG, temperature, etc.) as well as any manually entered, scanned, wirelessly or otherwise accessed data related to the patient, therapy, hospital, user, equipment, etc. The data can be uploaded, processed, stored, downloaded, and accessed/made available continuously, automatically, anytime significant data changes, when new data is input (e.g. arterial blood gas (ABG) data), on a fixed or variable time scale, or a combination thereof. The data may include all of the information available from one or more pieces of controlled equipment or other inputs, or it may be sampled, truncated, and/or parceled. The Database 200 may download/transmit information to any controlled equipment and/or directly operate the controlled equipment. Such direct operation may be in a semi-closed loop or closed loop modality.

Data within the Database 200 may be stored on a cloud server and/or locally (e.g. at the company) in order to allow for easy access. The Database 200 can continuously or discretely receive information to update the Database 200 and also continuously or discretely update any controlled equipment with historical and current information, as well as predictive therapies/diagnostics, equipment settings, therapy trends, prognosis, and concerns. The Database 200 may enable remote operation of one or more pieces of controlled equipment, allowing the user to be at a distance from the controlled equipment.

The Database 200 or any controlled equipment may sort, compile, and/or process available information and also enable tailored patient-specific therapy. Patient-specific therapy may take the form of inputting patient data and requesting or receiving outputs to direct or be used for patient therapy/diagnosis, including machine settings, medications, therapy/diagnostic timing and activities, forecasting, etc. The data processing and data outputs may be algorithm-driven and/or include the use of artificial intelligence/machine learning (AI/ML). For example, the Database 200 may receive historical/demographic patient data as a direct input from patient records or manually input which may include age, height, weight, sex, race/ethnicity, genetic map/genome information, medications, past therapies/conditions, etc. In addition, current or recent information such as current conditions/symptoms, ABG, cardiac related measurements (e.g. EKG), heart rate, blood pressure, and temperature can be input. This information may be processed by the Database 200 to provide outputs for patient care/equipment settings in general and as it relates to patients with similar characteristics, such as physical, diseases, conditions, treatment, etc. This output information can be tailored by choosing the input information and selecting the desired output information. The output information can provide the number of subjects the Database 200 evaluated/grouped, statistical relevance, any equipment settings (e.g. ventilation settings), medications, issues encountered with similar patients (e.g. adverse effects/events), etc. In addition, based on the input information, a statistical relevance may be selected and the Database 200 outputs the various ranges necessary to achieve that statistical relevance. Examples include age, sex, height, weight, ideal body weight (IBW), conditions, race/ethnicity, and genome that meet the desired statistical relevance. Those parameters may then be adjusted to see the effect on the statistics and sample size and resulting outputs for patient therapy/diagnosis, machine settings, medications, therapy/diagnostic timing and activities, forecasting, etc. Outputs may go to one or more of a Control Center 300, Handheld Communication Device 1300, or any controlled equipment. This can enable the user to make decisions based on specific patient data as well as data from a larger group of patients.

Database 200 information may be used in an open, semi-open, or closed loop operation to inform the user and/or direct changes to any controlled equipment. In open loop operation, information from the Database 200 may be displayed to the user with suggestions as to new settings, to remain at the current settings, or to provide guideline or warning/alarm information. In semi-open loop operation, information from the Database 200 may be used to make changes to certain selected parameters on controlled equipment without user input while other parameters will be provided to the user as suggestions. In closed loop operation, information from the Database 200 may be used to direct changes to controlled equipment and update displayed information on the user interface (UI) without requiring user input. In any of these scenarios, controlled equipment may be locked out of making certain selectable actions such as not going below a given threshold for any parameter. An example would be not allowing a Ventilator 500 in any operation (e.g. semi-closed loop or closed loop) to set the fraction of inspired oxygen (FiO2) below 21%. Any modes of operation may rely on sensors or external data sources to provide information to the Database 200 or controlled equipment to enable optimum ventilation.

### Apparatus for Data Display and Processing

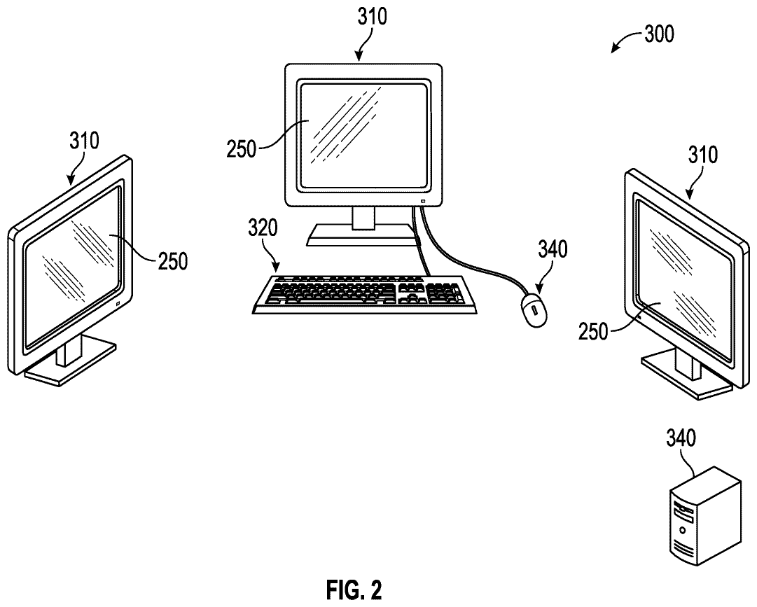

FIG. 2 illustrates an embodiment of a Control Center 300. The Control Center 300 can be used to receive, process, display, and/or send information to or from the Database 200 and/or controlled equipment and/or any other data that can be accessed or manually entered. The Control Center 300 may incorporate one or more Monitors 310, Keyboard 320, Mouse 330, and Computer 340. The Keyboard 320 and/or Mouse 330 may be used to input data, make selections, provide screen commands, any typical function of a computer keyboard and mouse. One or more Monitors 310 may be touchscreen, and as such can be used without or in association with the Keyboard 320 or Mouse 330, or the Keyboard 320 and Mouse 330 can serve as a primarily as a backup or be complimentary. The Computer 340 can function as the Database 200, as such the Computer 340 and Database 200 may be combined to provide inclusive functionality of both components. A Handheld Communication Device 1300 may serve as the Control Center 300.

The Control Center 300 may be local, at or near the patient, and/or remote, such as in a nursing or observation station within the hospital/care center or located in a different facility or physical location. Irrespective of location, the Control Center 300 may have the ability to communicate with other O2U System 100 components and controlled equipment. Communication may be wireless or wired, optical, etc. Having the Control Center 300 and/or controlled equipment in communication may allow for using the O2U System 100 for conducting treatment and/or diagnosis via telemedicine.

Data may be displayed on a Monitor 310, Handheld Communication Device 1300, and/or controlled equipment UI. Historical and current real-time data can be displayed as raw data, tables, charts, maps, including overlays with one or more parameters displayed over time and/or against each other. Displayed data can be adjusted in any axis (e.g. X-axis, Y-axis, or Z-axis) to show one or more parameters with visual indicators for in and/or out or nearly out of range parameters (e.g., data line or section of Display 250 is colored green when in normal range and red when out of normal range), trends and trend lines, changes in therapy (e.g., type and timing of medications, changes in ventilation settings) and the resulting effect(s), and forecasts. The parameters may be selected to include, but are not limited to, patient data (patient identifier, heart rate, blood pressure, temperature), controlled equipment data (ventilator settings and ventilation parameters; EKG; IV pump settings, medication(s) and fluid level/delivered fluid), other data (ABG, sensors) as well as local settings (local time, patient location, physician/staff). In addition to patient/treatment/equipment related data, the Display 250 can also provide from the Database 200 or Controlled Equipment 300 therapy guidelines and recommendations/best practice, such as hospital, local, regional, national, and global. These guidelines may be automatically or manually updated when released.

The Display 250 can update in any combination of continuously (e.g. EKG waveforms scrolling across a field in the Display 250), whenever a parameter changes, when there is new information available from the Database 200, or at user configurable times or amount of change(s).

The Display 250 may have fields which can be upgraded in software, without hardware changes, as this may allow rapid updates, modifications, configuration, and switching between/selecting languages (without having specific units for each region using/requiring a different language), even as optimum ventilation parameters change (e.g., during the COVID-19 crisis when best practices for ventilating patients rapidly changed).

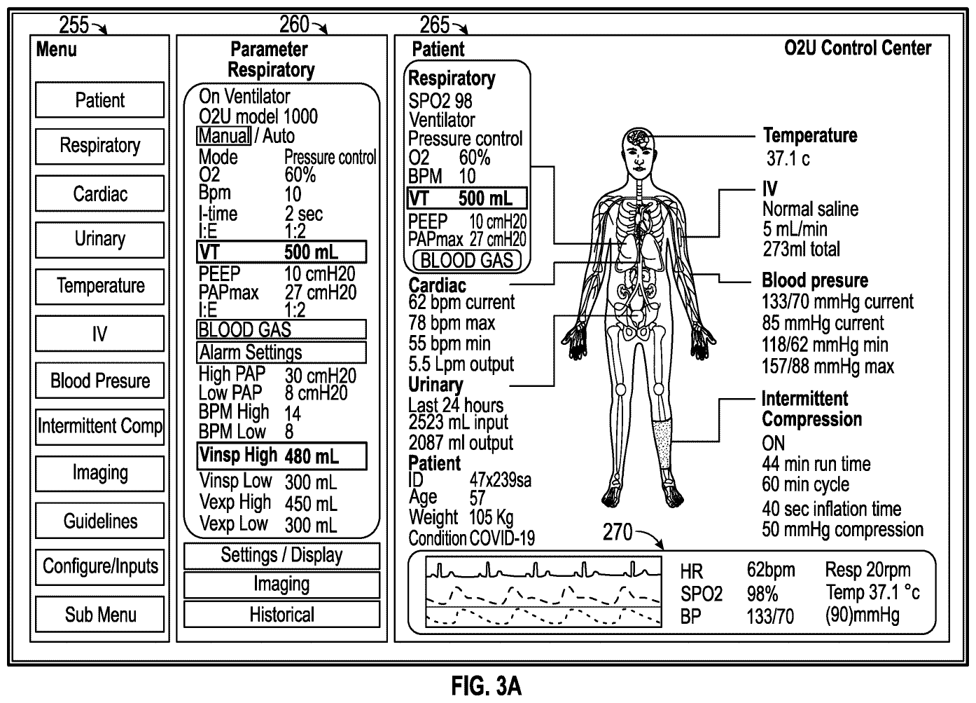

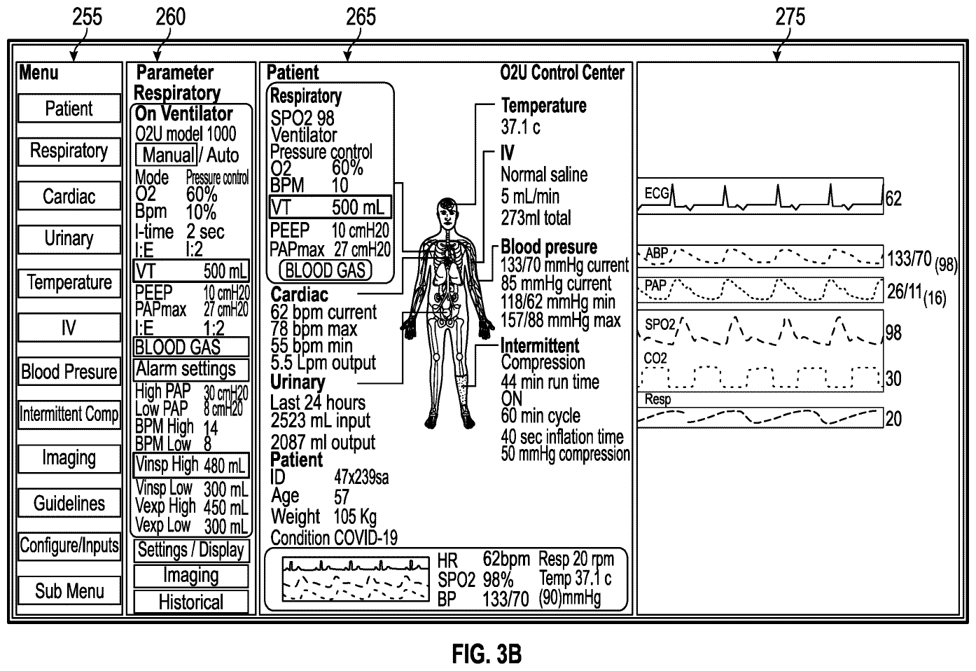

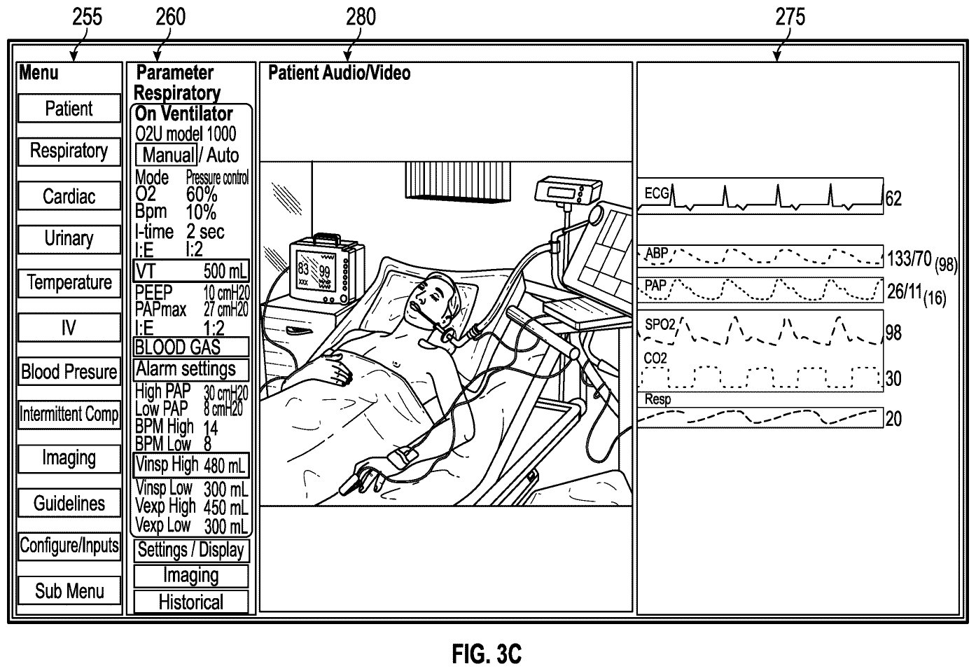

FIGS. 3A-3C illustrate examples of the Display 250. FIG. 3A illustrates an example of the Display 250. The Display 250 may contain one or more of, but is not limited to, the following fields: 1) a Menu Field 255 used for selecting different menu items, such as device and Display 250 configuration and layout, accessing sub-menus, inputs (data, patient, device, historical, current, other), guidelines, and parameter selection; 2) a Parameter Field 260, used for displaying parameters, such as, but not limited to, respiratory, cardiac, urinary, temperature, IV pump/medication, blood pressure, IPC, and imaging related data; 3) a Patient Field 265, used for displaying patient information/parameters, such as but not limited to data from one or more pieces of controlled equipment, an overview of data from selected parameters, imaging data, historical data, data from the Menu Field 255 and/or the Parameter Field 260, an input table or fields for inputting data, or any other data or field that may be selected; 4) Small Additional Parameter Field 270, which may contain data from any selected parameter, field, or menu item, e.g. the actual display or a portion thereof from one or more pieces of controlled equipment; 5) a Large Additional Parameter Field 275 which may contain data from any selected parameter, field, or menu item, e.g. the actual display or a portion thereof from one or more pieces of controlled equipment; and 6), an Audio/Visual Field 280, which has the ability to show the video image of the patient and enable communication with the patient.

Any of the above fields can be configured to be shown in different portions of the Display 250, by themselves on the Display 250, or in various combinations on the Display 250. An example is the Audio/Visual Field 280 in FIG. 3C, which is shown instead of the Patient Field 265 and Small Additional Parameter Field 270 in FIG. 3B. In the case of the Display 250 being on a Handheld Communication Device 1300, it may have only one or two fields shown at one time. In these types of configuration, when a selection is made, that field or data may replace the data presented on the Display 250, functioning similar to going to different pages.

Any of the Display 250 fields may show the screen or image(s) or representative screen of the actual user interface or display of a piece of controlled equipment or one that is viewed on the Audio/Visual Equipment 1400.

### Controlled Equipment

In some embodiments of the device and method described herein, the controlled equipment comprises a Ventilator 500. In some embodiments, the controlled equipment comprises general medical equipment such as cardiac monitors, IV Pump 1500, or blood gas analyzers; pulmonology equipment such as a ventilator and associated equipment; medical imaging equipment such as a fluoroscope, X-ray machine, ultrasound probe, intravascular ultrasound equipment; EKG or EEG equipment; or other equipment such as intermittent pneumatic compression (IPC), patient bed and bed settings (e.g. position), thermal (hot/cold), and pads/blankets. The controlled equipment may additionally comprise any Handheld Communication Device 1300 that is accessing or using one or more of the Database 200, O2U software, or communicating with other controlled equipment. The Database 200 may be contained within a piece of controlled equipment or be a separate entity.

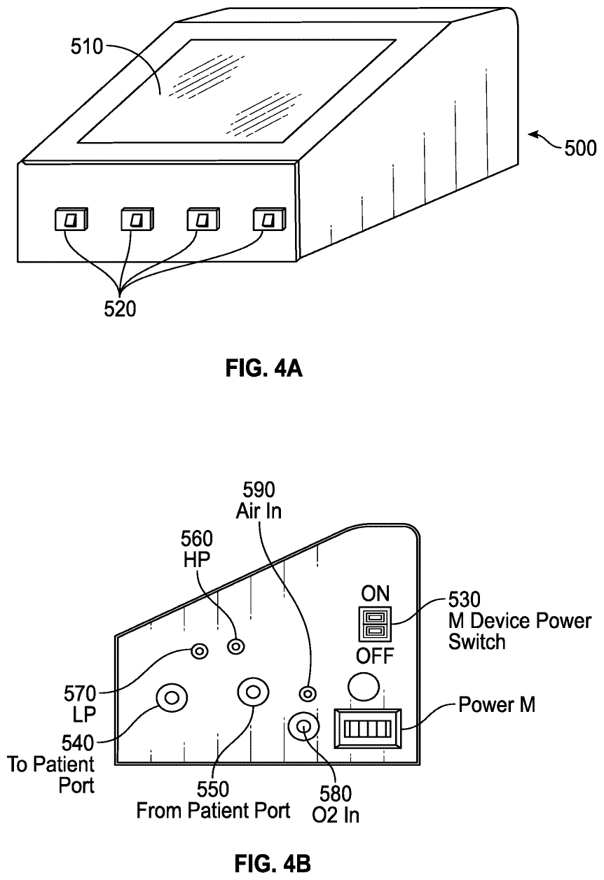

An example embodiment is shown in FIG. 4A, which illustrates in perspective view a type of controlled equipment, a Ventilator 500. FIG. 4B depicts a side view of the Ventilator 500. The Ventilator 500 may have software and/or hardware that uses an algorithm-driven workflow(s) and/or includes the use of AI/ML either internally or accessed through the Database 200 to provide for open, semi-open, or closed loop operation. The depicted Ventilator 500 contains a Ventilator UI 510 for the Display 250 and control and operation of many of the Ventilator's 500 features. The Ventilator UI 510 may include a touchscreen that may be detachable from the Ventilator 500. In this manner, the Ventilator UI 510 may also serve as a Handheld Communication Device 1300 and/or Control Center 300 which may be used to operate the Ventilator 500 ventilation functions at a distance physically away from the Ventilator 500 ventilation-related components (e.g., pump/compressor(s), valves, etc). This may be beneficial when the patient is in isolation or any situation where the user would need to don PPE to access ventilation controls, such as when dealing with a patient with a contagious disease (e.g., COVID-19). By having the ability to operate the Ventilator 500, or any of the controlled equipment and treat the patient from a distance, this may save not only the cost of PPE, but also may save the amount of time it takes the user to don and doff the PPE, allowing the user see/treat more patients during a given time period, with less risk of contamination to the user(s) and/or patient. In addition to the Ventilator UI 510, one or more Ventilator Switches 520 may be permanently on the Ventilator 500 itself, which may be beneficial in the event of a Ventilator UI 510 or communications error. Such Ventilator Switches 520 can be used to start, stop, and pause ventilation, switch ventilation modes, etc. A Power Switch 530 can also be a Ventilator Switch 520, used to turn on the mains or battery power to the Ventilator 500.

The Ventilator 500 may contain patient breathing circuit ports, a “To Patient” Port 540 and a “From Patient” Port 550. There may be one or more additional connections to the breathing circuit, a High Pressure Port 560 and a Low Pressure Port 570, for measuring airway pressures at the patient connection port of the breathing circuit. The Ventilator 500 may have an internal compressor or similar component to generate the pressurized air supply with oxygen coming from an external source. The oxygen supply can be connected to the Ventilator 500 through an Oxygen Inlet Connector 580. The Ventilator 500 may include an internal or external oxygen generator/concentrator to provide the oxygen supply. Optionally, there may also be an Air Inlet Connector 590 through which pressurized air can be delivered to the Ventilator 500. The Ventilator 500 may include a reservoir or input for anesthesia gas and be capable of delivering anesthesia gas at a user selectable or Database 200/controlled equipment determined rate. The Ventilator 500 can be directly or wirelessly connected to Audio/Visual Equipment 1400.

The Ventilator 500 may deliver and run in one or more of, but not limited to, the following breath types: Volume controlled, Pressure controlled, Pressure support, Spontaneous breaths and ventilation modes: Control, Assist/Control, SIMV, CPAP, BiPAP, Apnea Backup, Volume Pressure ventilation, and Bias Flow.

### Audio/Visual Equipment

Audio/Visual Equipment 1400 may include one or more cameras and one or more microphones, it may also include one or more speakers, controls to operate all the components, a patient interface, and image processing/analysis. The image processing/analysis may be located in the Database 200, in the Audio/Visual Equipment 1400, or in any of the controlled equipment, such as the Control Center 300. The Audio/Visual Equipment 1400 may enable the user to observe and speak to or communicate with the patient, have an interactive discussion with or visual/text through a patient interface, gauge respiration (e.g. identify dyssynchrony), gauge patient comfort/discomfort, determine patient position, observe the overall state of the patient, observe location/attachment of any sensors and/or equipment attached to or near the patient, and upload audio/visual information to the Database 200 or other equipment. The Audio/Visual Equipment 1400 may enable the user to provide the patient with certain instructions, such as telling the patient to conduct certain maneuvers during a test while allowing the user to visually observe how these are carried out, as well as generally interact with the patient. The patient interface may constitute a user interface, such as a tablet, handheld device, or headset that enables the user to communicate with the patient, through one or more of audio (if patient is capable), visual, and text (especially useful if the patient is intubated and cannot speak). The patient interface may incorporate use of a virtual reality headset. The Audio/Visual Equipment 1400 may be remotely adjustable, enabling the user to change camera position(s), zoom, focus, audio settings/controls (e.g. on, off, volume), etc. The visual capabilities may provide data for image processing/analysis via AI/ML, and can be used for but not limited to tracking and identifying patient movement, such as when to rotate patient, how long it has been since the last rotation/movement and display and track this information; breath rate and track and analyze breathing rates and correlate this with other patient parameters; comfort/discomfort, to analyze signs of comfort/discomfort using algorithms which may be trained on image data; signs of spontaneous breathing using algorithms which may be trained on breathing image data and measurements of breathing rates, patient-ventilation synchrony, identify and predict signs of spontaneous breathing; eliminate/minimize certain false alarms, such as the SpO2 alarm-if patient moves quickly (don't trigger alarm) or if the sensor if off the patient, inform the operator that the sensor is off as opposed to giving a low O2 saturation alarm; analyze ventilation parameters based on patient position (e.g., correlate with other data, blood gas, etc.), optimize parameters for each position and determine the patient's optimum position for ventilation.

### Remote Operation of the Device

Remote operation and/or interfacing with the controlled equipment, with or without audio/visual capabilities may be advantageous, both within an institution as well as controlled or evaluated from a more distant location. As previously described, remote operation of any of the controlled equipment can reduce contamination/spread of disease, as well as related PPE cost and the time involved with PPE. Remote operation enables an expert user/clinician to be at one location while the controlled equipment is at another location, potentially with a less trained/skilled/knowledgeable operator. In one embodiment, a physician or skilled operator is at a main control center located a distance away from the institution where the controlled equipment and patient are located. For example, a patient is being ventilated at a rural hospital by a respiratory technician or intern, while a pulmonologist critical care physician is looking at a Display 250 showing the same waveforms as shown on a Ventilator UI 510 of a controlled Ventilator 500. The physician is able to instruct the technician or intern on the proper settings, or directly control the Ventilator 500 to make the changes, for the patient's condition and ventilation, especially when the technician or intern's knowledgebase is such that they do not have the required information (knowledge) to determine the proper settings for a given condition. This capability is similarly useful when at the same institution, but the physician may be on call during afterhours. The controlled equipment may contain wired/wireless communications hardware and protocols to enable remote operation and/or interconnectivity between pieces of controlled equipment.

Another embodiment is the use of remote control/interfacing with controlled equipment is in a military/battlefield environment. The controlled equipment can, for example, be carried on a drone to the patient, where a medic or any other person in the field can connect the controlled equipment to the patient and be directed on how to operate the equipment and treat the patient from a remote location (e.g. base hospital), or have the controlled equipment operated remotely. The controlled equipment, for example a Ventilator 500, can have attachments/mounting points for connecting to and/or being powered by a drone or other military vehicle, as well as interfacing with military equipment and communications protocols, meeting the requirements for battlefield deployment (including physical and environmental conditions), and operation by military/related users.

### Overview of Workflow for Operation of Controlled Equipment

In some embodiments of the device and method disclosed herein, the controlled equipment has software and/or hardware that uses (an) algorithm-driven workflow(s) and/or includes the use of AI/ML ether internally or accessed through the Database 200 to provide or open, semi-open, or closed loop operation. Algorithm-based operation may allow for one or more algorithms to be used to inform and/or direct the user and/or directly control the operation of controlled equipment with or without user input. In some embodiments, the workflow is driven by the equipment wherein the Control Center 300 displays and controls to operate and receive data from multiple pieces of equipment such as a cardiac monitor, ventilator, blood gas analyzer, IV pump(s), intermittent pneumatic compression (IPC), patient bed and bed settings (e.g. position), thermal (hot/cold) pads/blankets, or others. Such pieces of equipment may be wired, wireless, or some combination thereof.

In an example, the Ventilator 500 has software and/or hardware that uses (an) algorithm-driven workflow(s) and/or includes the use of AI/ML either internally or accessed through the Database 200 to provide for open, semi-open, or closed loop operation. Algorithm-based operation may allow for one or more algorithms to be used to inform and/or direct the user and/or directly control the operation of controlled equipment with or without user input. One or more algorithms may be used for configuring baseline ventilation parameters and corresponding alarm settings adjusted for those baseline ventilation settings, as well as initiating and continuing ventilation of the patient, detecting and adjudicating alarms as well as determining alarm false positives, running specific tests (e.g., plateau pressure) and/or monitoring parameters to determine the success of ventilation, trends, and determining when a patient is ready to be weaned from the Ventilator 500 as well as determining successful or unsuccessful weaning.

Integration of AI/ML Workflows with Device

Artificial intelligence and machine learning (AI/ML) can be used in/with one or more pieces of controlled equipment, as well as the Database 200. Examples include the Control Center 300, Ventilator 500, and Audio/Visual Equipment 1400. The Database 200 and/or controlled equipment may have all individual patient data accessible as well as a database of all patients and may sort, compile, and/or processes available information and also enable tailored patient-specific therapy using at least in part AI/ML. Patient-specific therapy may take the form of inputting patient data and requesting or receiving outputs to direct or be used for patient therapy, including machine settings, therapy/diagnostic timing and activities (e.g. medications, ventilation parameters, position, diagnostic tests) forecasting, etc. For example, the Database 200 can receive historical/demographic patient data as a direct input from patient records or manually input which may include age, height, weight, sex, race, genetic map/genome information, medications, past therapies/conditions, etc. In addition, current or recent information such as arterial blood gas (ABG), cardiac related measurements (e.g. EKG), heart rate, blood pressure, and temperature can be input. This information can be processed by the Database 200 to provide outputs for patient care/equipment settings as it relates to patients with similar demographics, disease, conditions, treatment, etc. This output information can be tailored by choosing the input information and selecting the desired output information. The output information can provide the number of subjects evaluated/grouped, statistical relevance, any equipment settings (e.g. ventilation settings, medications), issues encountered with similar patients, etc. In addition, based on the input information, a statistical relevance may be selected, and the Database 200 may output the various ranges, which may include age, sex, height, weight, IBW, conditions, race that meet the desired statistical relevance. Those parameters may then be adjusted to see the effect on the statistics and sample size and resulting outputs for patient therapy, machine settings, therapy/diagnostic timing and activities, forecasting, etc. Outputs may go to one or more of a Control Center 300, Handheld Communication Device 1300, or controlled equipment. This may enable the user to make decisions based on specific patient data, data from a larger group of similar patients (e.g. demographics and/or condition(s)), and/or data from all patients.

### Machine Learning Models

Described herein are models that inform providing respiratory support to a patient. In some cases, the models may optimize performance of a ventilator, predict patient outcomes, optimize therapy/diagnostic timing and activities, forecast, and the like. Additionally, in some cases, the models may be used to optimize the performance of multiple ventilators. In some instances, the optimization is determined by comparing an input comprising patient parameters to a developed model. The model may comprise a machine learning algorithm. In some cases, the machine learning algorithm structure may be alternative decision trees (ADTrec), Decision Stumps, functional trees (FT), logistic model trees (LMT), logistic regression, supper vector machines (SVM), neural network, k-means clustering or any machine learning algorithm or statistical algorithm known in the art.

In some cases, the machine learning model may be created by training a machine learning algorithm with a data set of patients' parameters and respective ventilation parameters, patient outcomes, treatment regimens, and the like. More specifically, by way of nonlimiting example, parameters comprising input and/or output may comprise patient data comprising demographic information, disease information, conditions, or treatments; arterial blood gas measurements (ABG; blood pressure; body temperature; heart rate; respiration; general health; patient position; respiratory rate; tidal volume (VT); minute ventilation; PEEP; pH; PaO2/FiO2; pulmonary shunt fraction; negative inspiratory forces; frequency; stable cardiovascular status; hemoglobin; systolic blood pressure; vasopressor or inotrope; trigger sensitivity; flow rate; waveform; and inspiratory/expiratory (I/E) ratio; images; audio; and videos. In some cases, training may be supervised training. Alternatively, training may be unsupervised training. In some instances, the data set of patients' parameters and output data may be a retrospective data set. Alternatively, the data set of patients' parameters may be a prospectively developed dataset, and the machine learning model may be iteratively improved over time.

FIG. 6 illustrates a schematic diagram of a data processing workflow 700 to train a machine learning model with a data set of patients' parameters that may provide outputs to optimize performance of a ventilator. The data processing workflow 700 generally comprises the steps of preprocessing 730, training 740, and predicting 770. The data processing workflow may extract training data 710 from a database, or intake new data 720. The preprocessing step 730 may apply one or more transformations to standardize the training data or new data for the training step of the prediction step. The preprocessed training data may be passed to the training step 740, which may construct a machine learning model 750 based on the training data. The training step may further comprise a validation step 760, configured to validate the trained machine learning model using any appropriate validation algorithm (e.g., Stratified K-fold cross-validation). The preprocessed new data may be passed on to the predicting step 770, which may output a prediction 780 of ventilation parameters, predicted patient outcomes, therapy/diagnostic timing or activities, forecasting, or the like.

The training data 710, used by the training step to construct the machine learning model, may comprise a plurality of patient parameter datasets from a plurality of patients, each patient's dataset comprising an array of parameters as previously described herein. For example, as described here, the parameters may include patient data comprising demographic information, disease information, conditions, or treatments; ABG; blood pressure; body temperature; heart rate; respiration; general health; patient position; respiratory rate; VT; minute ventilation; PEEP; pH; PaO2/FiO2; pulmonary shunt fraction; negative inspiratory forces; frequency; stable cardiovascular status; heart rate; hemoglobin; systolic blood pressure; vasopressor or inotrope; trigger sensitivity; flow rate; waveform; and inspiratory/expiratory (I/E) ratio; images; audio; and videos. The training data may comprise datasets available from large data repositories. The training data may comprise patient data from healthy patients, as well as patient data from a population of patients on ventilators. The patient data may comprise diagnoses, therapy results, other patient outcomes, and the like.

The preprocessing step 730 may apply one or more transformations to the training data to clean and normalize input patient parameter data, for example. The preprocessing step may be configured to discard parameters which contain spurious data or contain very few observations. The preprocessing module can be further configured to standardize the encoding of parameter values. Different input patient parameter datasets may often have the same parameter value encoded in different ways, depending on the source of the dataset. For example, ‘900’, ‘900.0’, ‘904’, ‘904.0’, ‘−1’, ‘−1.0’, ‘None’, and ‘NaN’ may all encode for a “missing” parameter value. The preprocessing step may recognize the encoding variation for the same value and standardize the dataset to have a uniform encoding for a given parameter value. The preprocessing step may thus reduce irregularities in the input data for the training and prediction steps, thereby improving the robustness of the training and prediction steps.

The preprocessing step may impute any missing input parameter data values, such that downstream steps may correctly process the data. For example, if a training dataset provided to the training step is missing a parameter value, the preprocessing step may provide the missing value, so that the dataset may be processed correctly by the training step. Similarly, if a new patient parameter dataset provided to the prediction step is missing one or more feature values, the preprocessing step may provide the missing values, so as to enable correct processing of the dataset by the prediction step.

The training step 740 may utilize a machine learning algorithm or other algorithm to construct and train a machine learning model to be used in the determination of ventilation parameters, suggest a course of treatment, predict a patient outcome, or the like. The machine learning model may, for example, comprise the statistical correlations between a plurality of patient parameters indicative of a set of optimal parameters for a ventilator.

One or more machine learning algorithms may be used to construct the machine learning model, such as support vector machines that deploy stepwise backwards parameter selection and/or graphical models, both of which may have advantages of inferring interactions between parameters. For example, machine learning algorithms or other statistical algorithms may be used such as alternating decision trees (ADTree), Decision Stumps, functional trees (FT), logistic model trees (LMT), logistic regression, Random Forests, receiver operational characteristic curves (ROC), linear regression, or any machine learning algorithm or statistical algorithm known in the art. One or more algorithms may be used together to generate an ensemble method, wherein the ensemble method may be optimized using a machine learning ensemble meta-algorithm such as boosting (e.g., AdaBoost, LPBoost, TotalBoost, BrownBoost, MadaBoost, LogitBoost, etc.) to reduce bias and/or variance. Once a machine learning model is derived from the training data, the model may be used to provide respiratory support for a patient by optimizing performance of a ventilator, suggestions for patient therapy, machine or equipment settings, therapy/diagnostic timing and activities, forecasting, and the like. Machine learning analyses may be performed using one or more of many programming languages and platforms known in the art, such as R, Weka, Python, and/or MATLAB, for example.

### Tuning Machine Learning Model Features

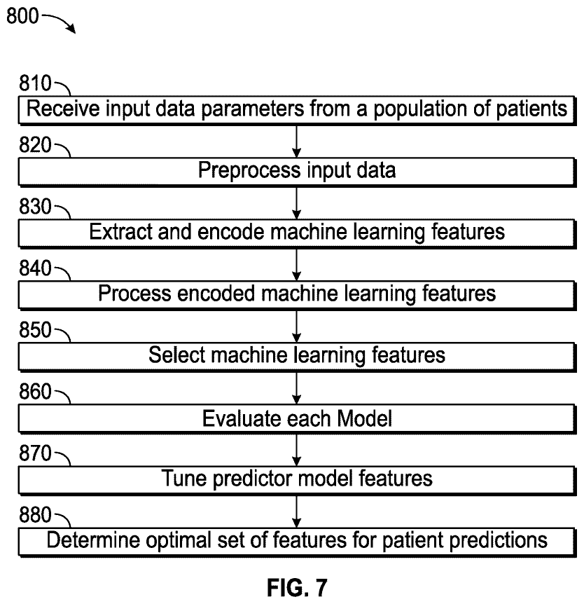

FIG. 7 is a method of tuning parameters of a predictive machine learning model, as described, herein based on patient data resulting in a predictive model configured to provide respiratory support to a patient. As shown in FIG. 7, a method 800 is provided for using machine learning algorithms to train a machine learning model and tune its configuration parameters optimally. Multiple machine learning predictive models may be trained and tuned using the method 800 for an input parameter from a population of patients 810. The model training method preprocesses data from a plurality of patients using machine learning techniques at 820. Datasets may be pre-processed using well-established machine learning techniques such as data cleaning, filtering, aggregation, imputation, normalization, and other machine learning techniques as known in the art.

The model training method extracts and encodes machine learning features from the preprocessed patient data at 830. Columns comprising the parameter datasets may be mapped into machine learning features using feature encoding techniques such as, for example, one-hot encoding, severity encoding, or any other feature encoding technique as known in the art.

The model training method processes the encoded machine learning features at 840. In some instances, the patient parameter data may be encoded into machine learning features, after which, a sample weight may be computed and assigned to every patient parameter in a data set. Samples may be grouped according to patient-specific dimensions and sample weights may be computed and assigned to balance one group of samples against every other group of samples to mirror the expected distribution of patients.

The model training method selects a subset of the processed machine learning features at 850. In some cases, with the training samples weighted accordingly, and all potential machine learning features encoded appropriately, feature selection may take place a machine learning process generally known as bootstrapping, where multiple iterations of model training can be run, each using a random subsample of training data available. After each run, a tally can be updated with the features the training process deemed necessary to include in the model. This list can be expected to vary from run to run, since the random data subsets used in the training might contain apparent patterns that are incidental to the choice of data samples and not reflective of real-life patterns. Repeating this process multiple times may allow for the incidental patterns to cancel out, revealing the features that are reflective of patterns that can be expected to generalize well outside the training data set and into the real world. The top features of the bootstrapping runs can then be selected and used exclusively for training the final model, which is trained using the entire training data set, and saved for later application.

The model training method may evaluate each model at 860. In particular, each model may be evaluated for performance, for example, as determined by sensitivity and specificity for a pre-determine inclusion rate. In some cases, using a held-out patient parameter data set that was not used during the model training phase, the models may be evaluated for performance, in terms of inclusion rate, sensitivity, and specificity.

The model training method may tune each predictor model parameters at 870. More specifically, to assess the performance of the models in different tuning settings, the tuning parameters of each model may be changed in iterative increments and the same metrics may be computed over the same held-out set in every iteration. The optimal setting may then be locked in and corresponding models saved. Tuning parameters may include, for example, the number of layers and nodes in a neural network, the number of trees in a boosted decision tree model, the maximum depth of every tree, the learning rate, the threshold of positive determination score, the range of output deemed inconclusive, and any other tuning parameter as known in the art.

In some instances, the parameter tuning process 870 may comprise a brute-force grid search, an optimized gradient descent or simulated annealing, or any other space exploration algorithm as known in the art. The models being tuned may undergo separate, independent tuning runs, or alternatively the models may be tuned in an ensemble approach, with every parameter of every model explored in combination in order to arrive at the optimal overall set of parameters at Y08 to maximize the benefit of using all the models in an ensemble.

### Machine Learning Model Associations

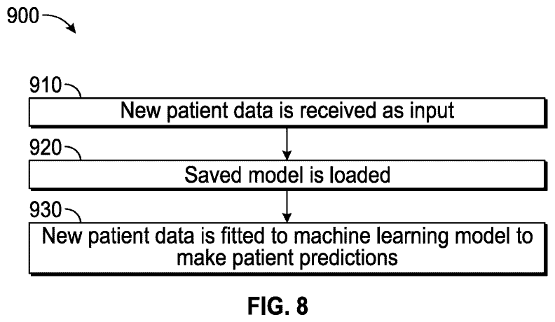

FIG. 8 shows an operational flow 800 of a method implementing a machine learning model configured to provide respiratory support to a patient. The machine learning model 750, described herein may be configured to generate an association and prediction for a patient, by fitting new data to a machine learning model constructed in a training step 740. At step 910, the machine learning model may receive new data that may have been processed by the preprocessing step 730 previously described, to standardize the data, for example by dropping spurious metadata, applying uniform encoding of feature parameter values, re-encoding select features using different data representations, and/or imputing missing data points, as described herein. The new data may comprise an array of patient parameters for a particular patient. The new data my comprise a previously collected, complete parameter data set for a patient to provide respiratory support to the patient.

At step 920, the method may load a previously saved machine learning model, constructed by the training step, from a local memory and/or a remote server configured to store the model. At step 930, the new patient parameter data is fitted to the machine learning model to generate outputs that provide respiratory support to the patient. Such outputs, by way of nonlimiting example, may comprise patient therapies, ventilator settings or ventilation parameters, other machine parameters or settings, therapy/diagnostic timing and activities, forecasting, and the like. In some embodiments, by way of nonlimiting example, the outputs comprise ventilator settings comprising FiO2, frequency or respiratory rate, peak flow rate, flow pattern, timing, peak inspiratory pressure (PIP), PEEP, pressure support (PS), and VT.

### Computer Systems

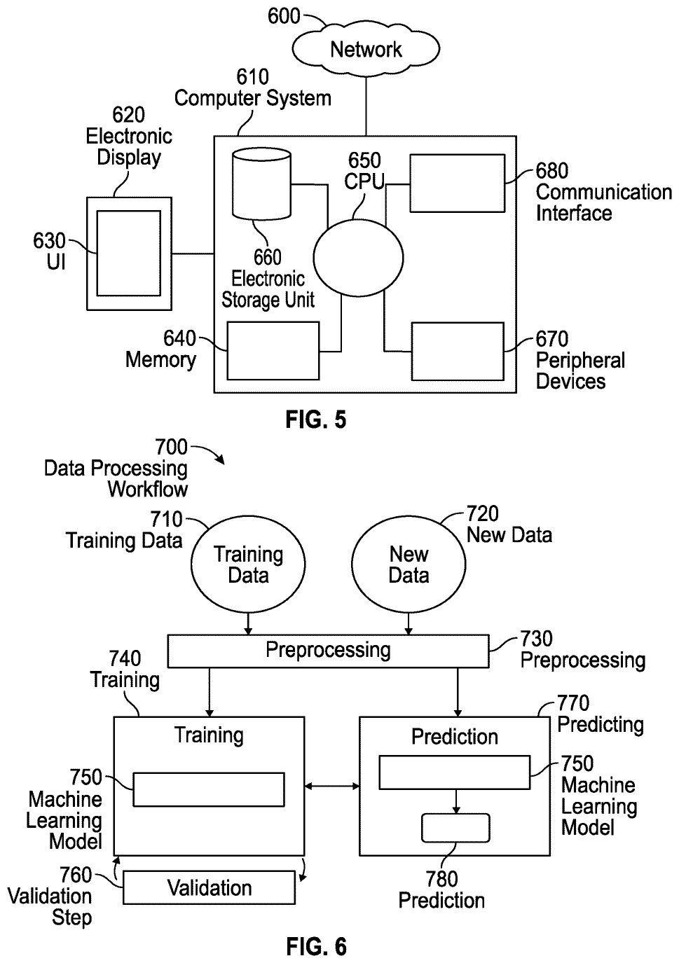

FIG. 5 shows a computer system 610 suitable for implementing the machine learning model methods described herein. The computer system 610 may process various aspects of information of the present disclosure, such as, for example, patient input parameters and statistical analysis. The computer system 610 may be an electronic device. The electronic device may be a mobile electronic device.

The computer system 610 may comprise a central processing unit (CPU, also “processor” and “computer processor” herein) 650, which may be a single core or multi-core processor, or a plurality of processors for parallel processing. The computer system 610 may further comprise memory or memory locations 640 (e.g., random-access memory, read-only memory, flash memory), electronic storage unit 660 (e.g., hard disk), communication interface 680 (e.g., network adapter) for communicating with one or more other devices, and peripheral devices 670, such as cache, other memory, data storage and/or electronic display adapters. The memory 640, storage unit 660, communication interface 680, and peripheral devices 670 are in communication with the CPU 650 through a communication bus (solid lines), such as a motherboard. The storage unit 660 may be a data storage unit (or a data repository) for storing data. The computer system 610 may be operatively coupled to a computer network (“network”) 600 with the aid of the communication interface 680. The network 600 may be the Internet, an internet and/or extranet, or an intranet and/or extranet that is in communication with the Internet. The network 600 may, in some case, be a telecommunication and/or data network. The network 600 may include one or more computer servers, which may enable distributed computing, such as cloud computing. The network 600, in some cases with the aid of the computer system 610, may implement a peer-to-peer network, which may enable devices coupled to the computer system 610 to behave as a client or a server.

The CPU 650 may execute a sequence of machine-readable instructions, which may be embodied in a program or software. The instructions may be directed to the CPU 650, which may subsequently program or otherwise configured the CPU 650 to implement methods of the present disclosure. Examples of operations performed by the CPU 650 may include fetch, decode, execute, and writeback.

The CPU 650 may be part of a circuit, such as an integrated circuit. One or more other components of the system 610 may be included in the circuit. In some cases, the circuit is an application specific integrated circuit (ASIC).

The storage unit 660 may store files, such as drivers, libraries and saved programs. The storage unit 660 may store patient parameter data, e.g., height, weight, age, etc. and programs that act upon the donor parameter data. The computer system 610, in some cases may include one or more additional data storage units that are external to the computer system 610, such as located on a remote server that is in communication with the computer system 610 through an intranet or the internet.

Methods as described herein may be implemented by way of machine (e.g., computer processor) executable code stored on an electronic storage location of the computer device 610, such as, for example, on the memory 640 or electronic storage unit 660. The machine executable or machine-readable code may be provided in the form of software. During use, the code may be executed by the processor 650. In some instances, the code may be retrieved from the storage unit 660 and stored on the memory 640 for ready access by the processor 650. In some instances, the electronic storage unit 660 may be precluded, and machine-executable instructions are stored on memory 640.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 610, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor and the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage’ media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards, paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 620 that comprises a user interface (UI) 630 for inputting patient parameters and viewing the results of modelling. Examples of UI's include, without limitation, a graphical user interface (GUI) and web-based user interface. In some embodiments, the Computer 340 may comprise the computer system 610 while in other embodiments the Computer 340 and the computer system 610 may be separate.

Methods and systems of the present disclosure can be implemented by way of one or more algorithms and with instructions provided with one or more processors as disclosed herein. An algorithm can be implemented by way of software upon execution by the central processing unit 650. The algorithm can be, for example, random forest, graphical models, support vector machine, neural network, or other.

### Data Input and Preliminary Determination of Parameters

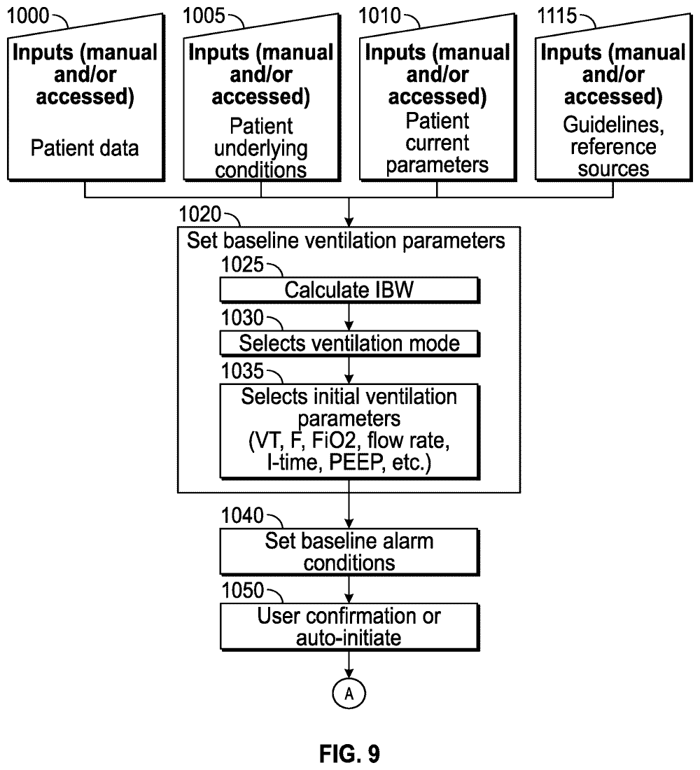

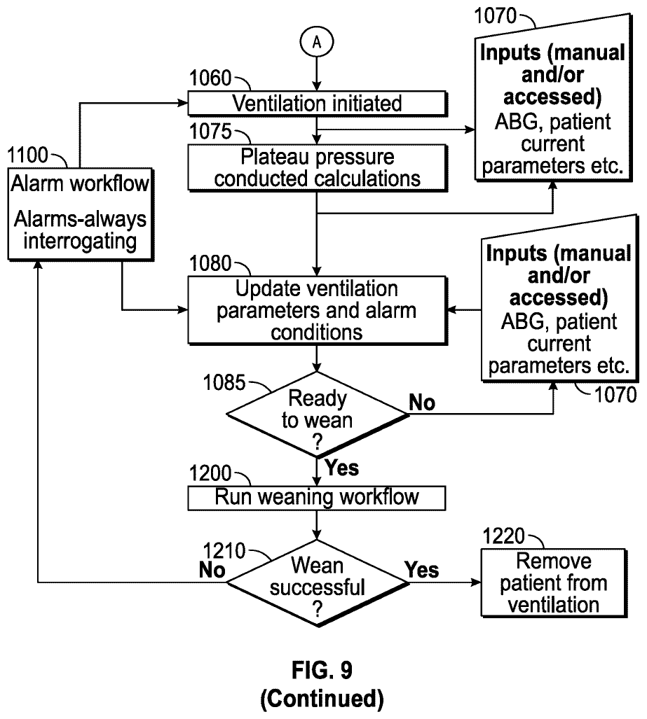

FIG. 9 illustrates an embodiment of an algorithm-based workflow. Example inputs include one or more of the following: Input-Patient Data 1000, Input-Patient Underlying Conditions 1005, Input-Patient Current Parameters 1010, and Input-Guidelines, Reference Sources 1015. Input-Patient Data 1000 may comprise data related to the patient's age, height, weight, sex, race, etc. Input-Patient Underlying Conditions 1005 may comprise respiratory related conditions/status, such as normal/current respiratory function, chronic obstructive pulmonary disease (COPD), acute lung injury (ALI), acute respiratory distress syndrome (ARDS), brain injury, smoker or non-smoker, as well as other health disorders and diseases, such as diabetes, cardiac disease, neuro-muscular disease, kidney disease, etc. Input-Patient Current Parameters 1010 may comprise the current parameters such as arterial blood gas measurements (ABG), blood pressure, body temperature, heart rate, respiration rate, general health (e.g. cold, flu, cough), etc. Patient position can serve as a parameter that is tracked and monitored to determine the optimum ventilation parameters for a given position (supine, prone, etc.). Input-Guidelines, Reference Sources 1015 may comprise any related applicable guidelines and reference data, such as that from ARDSnet, the American Association for Respiratory Care (AARC) guidelines, the NIH COVID-19 Treatment Guidelines, state, local and institutional guidelines and best practices, etc.

All or a subset of these inputs may be used to set the patient's Baseline Ventilation Parameters 1020. The patient's height, weight, and sex can be used to calculate the patient's IBW: Calculate Ideal Body Weight 1025. A typical formula used is IBW for males (kg)=50+[0.9×(Height(cm)−154)] and IBW for females (kg)=45.5+[0.9×(Height(cm)−154)]. The patient's IBW, age, guidelines, and other factors may be used to Select Breath Type/Ventilation Mode 1030 from a lookup table, which may be locally accessed within the Ventilator 500 or accessed from the Database 200. The lookup table may provide selection criteria for breath type, mainly selecting from: Assist-control (AC) with pressure or volume target, Volume-controlled (VC), pressure-controlled (PC), or pressure regulated volume control ventilation (PRVC), and synchronized intermittent mandatory ventilation (SIMV) may be added to one of the other modes as well. Institutions may prefer either pressure or volume as the set point. In this case, the Ventilator 500 or Database 200 can be programmed according to each institution or even each user. However, the lookup table may additionally or alternatively select PC ventilation for patients with hypoxia or poor lung compliance, neonates, and infants, whereas VC may be selected for patients larger than infants with more normal lung compliance. AC with pressure or volume target, or SIMV may be selected for COPD obtunded or postoperative patients. The breath type and optionally the information why that breath type was selected may be presented to the user or directly set as one of the Baseline Ventilation Parameters 1020 depending on the operational mode of the Ventilator 500 (e.g., open, semi-open, or closed loop operation).

All or any subset of the inputs may be used to determine the patient's Initial Ventilation Parameters 1035. The Ventilator 500 or Database 200 can present or set one or more of the following, or still other, parameters: tidal volume (VT), frequency (respiratory rate in breaths per minute (bpm)), FiO2, breathing gas flow rate, inspiration time (I-time), peak end-expiratory pressure (PEEP), and apnea. VT is determined using age, respiratory function/disease, and IBW. The value for VT in adult patients with normal lungs is 8-14 mL/kg, more preferably 10-12 mL/kg ideal body weight, with COPD 8-12 mL/kg, more preferably 8-10 mL/kg ideal body weight, with ARDS 4-10 mL/kg, more preferably 6-8 mL/kg ideal body weight, and then infants are at 5-12 mL/kg, more preferably 7-10 mL/kg, and neonates are 2-8 mL/kg, more preferably 4-6 mL/kg. Frequency may be based off of age and respiratory function/disease. The value for frequency in adult patients with normal lungs is 8-20 bpm, more preferably 10-12 bpm, with COPD 10-18 bpm, more preferably 12-14 bpm, with ARDS 14-22 bpm, more preferably 16-18 bpm, and then adolescents at 10-18 bpm, more preferably 12-16 bpm, infants at 15-40, mor preferably 20-30 bpm, and neonates at 20-50 bpm, more preferably 30-40 bpm. FiO2 is typically initiated between 40-100%, with 100% being used for most cases except when a relatively healthy patient is being ventilation, such as during routine surgery, and not when there are potential or actual breathing/respiratory issues. Flow rate is generally 20-80 L/min, more preferably 40-60 L/min. I-time for adults is 0.5-2.0 sec, more preferably 1.0-1.5 sec. For pediatrics, an inspiratory to expiratory ratio (I:E) is 1:1.5-1:3 is used, more preferably 1:2, in addition, in PC, the Broselow or PALS manual 10 settings can be used. PEEP ranges from 0-10 cmH2O, more preferably 0-5 cmH2O, except in patients presenting with COPD, then the initial range is 3-10 cmH2O, more preferably 5-8 cmH2O. Apnea (apnea backup) values are based on the bpm selected. If the selected bpm is >12, the apnea rate is set to the bpm, if the selected bpm <12, the apnea rate is set to 12 bpm if not rate limited to <12 bpm by another input, if the selected bpm is <12 and rate limited to <12 bpm, the apnea rate is set to the maximum allowable bpm rate. The use may then then confirm/accept the Baseline Ventilation Parameters 1020 of wait until after the Baseline Alarm Conditions 1040 are set.

### Selection of Alarm Conditions

Once the Baseline Ventilation Parameters 1020 are determined, the Ventilator 500 or Database 200 can present or set one or more of the selected Baseline Alarm Conditions 1040. The Baseline Alarm Conditions 1040 may be set in relation to the Baseline Ventilation Parameters 1020 by the Ventilator 500 or Database 200, with the alarm values tailored to the ventilation settings. In an example, a high frequency alarm is used for a neonate where the selected frequency is 35 bpm. Using a baseline plus 10% and rounding up, the high frequency alarm would be set to 39 bpm. In another example, for a normal adult with a selected frequency of 10 bpm, a larger percentage, e.g., 40%, is used, such that the high frequency alarm would be set to 14 bpm. These alarm settings may be selected based on baseline plus/minus a fixed or variable amount, or fixed or variable percentage, or any combination thereof, sometimes with an absolute maximum or absolute minimum depending on the parameter.

The formulas/algorithms used for selecting baseline values for the alarms may include, but are not limited to, the following: The high airway pressure alarm may be set using baseline plus a fixed amount (e.g., the alarm set point would be baseline plus 5-20 cmH2O with an absolute maximum of 40 cmH2O, whichever is lower, more preferably baseline plus 5-10 cmH2O or 40 cmH2O, whichever is lower). High airway pressure may be set using a percentage formula (e.g., the alarm set point would be baseline plus 10-20% or a minimum value of baseline plus 2-10 cmH2O, more preferably baseline plus 15-20% with a minimum value of baseline plus 3-6 cmH2O and a maximum value of 40 cmH2O). For example, with a baseline peak airway pressure of 25 cmH2O, using baseline plus 5 cmH2O, the alarm set point would be 30 cmH2O. Using baseline plus 15% and rounding up, the alarm set point would be 29 cmH2O. If a minimum over baseline requirement was employed and set at 5 cmH2O, this 29 cmH2O would be increased to 30 cmH2O.

In some cases, the low airway pressure alarm may be set using a baseline minus a fixed amount. In an example, the alarm set point may comprise baseline minus 5-20 cmH2O with an absolute minimum of either 0-5 cmH2O or the set PEEP, whichever is higher, such as baseline minus 5-10 cmH2O with an absolute minimum of either 0-5 cmH2O or the set PEEP, whichever is higher. In some cases, the low airway pressure may be set using a percentage formula. In an example, the alarm set point may comprise baseline minus 10-50% or a minimum value of baseline minus 2-10 cmH2O with an absolute minimum of either 0-5 cmH2O or the set PEEP, more preferably baseline minus 15-20% with a minimum value of baseline minus 3-6 cmH2O with an absolute minimum of either 0-5 cmH2O or the set PEEP. In another example, with a baseline minimum airway pressure of 8 cmH2O and a PEEP of 5 cmH2O, using baseline minus 4 cmH2O, the alarm set point would be 8 cmH2O minus 5 cmH2O equaling 3 cmH2O. However, under the PEEP constraint of a minimum of 5 cmH2O, the low airway pressure alarm would be set at 5 cmH2O. In another example, using baseline minus 50% with the PEEP set to 0 cmH2O and a baseline minimum airway pressure of 8 cmH2O, the alarm set point would be 8 cmH2O minus 4 cmH2O, equaling 4 cmH2O. In this case, the set point is above the PEEP, so the alarm would be set at 4 cmH2O.

In some cases, a low expired volume alarm may be determined using baseline minus a fixed amount. The alarm set point may comprise baseline minus 5-200 mL, with an absolute minimum depending on the patient age, weight, target VT, minute rate, or other parameters, such as baseline minus 25-100 mL for a normal adult down to baseline minus 2-20 mL or more preferably baseline minus 2-5 for a neonate. In some cases, the low expired volume alarm may be determined using a percentage formula. In such cases, the alarm set point may comprise baseline minus 5-50% with an absolute minimum depending on the patient age, weight, or other parameters and target VT, such as baseline minus 5-25% for certain patient groups. For example, with a baseline VT of 500 mL in an adult, using baseline minus 50 mL, the alarm set point would be 450 mL. Using baseline minus 15% with a baseline VT of 500 mL, the alarm set point would be 425 mL. In a neonate with a baseline of 20 mL, baseline minus 20% would give an alarm set point of 16 mL. Examples of absolute minimums are, for a normal adult with a VT over 500 mL, 150-300 mL, more preferably 200-250 mL. For a neonate with a VT of 20 mL, the minimum could be as low as 10-15 mL.

In some cases, a high frequency alarm may be determined from a baseline plus a fixed amount. In such cases, the alarm set point may comprise baseline plus 1-30 bpm with an absolute maximum of 80 bpm, whichever is lower, such as baseline plus 1-5 bpm in a normal adult with a maximum of baseline plus 10-20 bpm, ranging to baseline plus 5-15 bpm in a neonate with a maximum of baseline plus 10-30 bpm. In some cases, a high frequency alarm may be set using a percentage formula. In such cases, the alarm set point would be baseline plus 10-100%, with an absolute maximum of 80 bpm, whichever is lower, such as baseline plus 20-80% in a normal adult ranging to baseline plus 10-50% in a neonate. For example, with a baseline frequency of 10 bpm in a normal adult, using baseline plus 5 bpm, the alarm set point would be 15 bpm. Using baseline plus 40%, the alarm set point would be 14 bpm. In a neonate where the baseline frequency is 35 bpm, using a baseline plus 10% and rounding up, the high frequency alarm would be set to 39 bpm. Another operation may comprise determining the amount over baseline by dividing baseline frequency into groups and then providing a different amount over baseline for each group. The baseline frequencies can be divided into two or more groups. In an example, for baseline frequencies >20 bpm, such as a neonate with a selected frequency of 35 bpm, the high frequency alarm may be set to baseline plus 10%, rounded up to give 39 bpm. In some cases, baseline frequencies may comprise <20 bpm, such as a normal adult with a high frequency setting of 10 bpm. In such cases, the high frequency alarm may be set to baseline plus 40%, with a resulting high frequency alarm setting of 14 bpm.

The apnea alarm may be triggered when the Ventilator 500 meets the apnea criteria and goes into apnea backup. In an example, the apnea criteria may comprise: if the selected bpm is ≥12, the apnea rate is set to the bpm; if the selected bpm <12, the apnea rate is set to 12 bpm if not rate limited to <12 bpm by another input If the selected bpm is <12 and rate limited to <12 bpm, the apnea rate is set to the maximum allowable bpm rate. Other apnea criteria are possible. The default for the apnea audio alarm may be set to silent with just a visual indicator of the Ventilator 500 operating in apnea backup.

In some cases, a high PEEP alarm may be set using baseline plus a fixed amount. In such cases, the alarm set point may comprise baseline plus 0-20 cmH2O with an absolute maximum of typically 40 cmH2O, whichever is lower, such as baseline plus 0-10 cmH2O or 40 cmH2O, whichever is lower. Alternatively, high PEEP alarm may be determined using a percentage formula. In such cases the alarm set point may comprise baseline plus 0-200% or a minimum value of baseline plus 2-10 cmH2O, such as baseline plus 40-150% with a minimum value of baseline plus 3-6 cmH2O and a maximum value of 40 cmH2O. For example, with a baseline PEEP of 5 cmH2O, using baseline plus 5 cmH2O, the alarm set point would be 10 cmH2O, using baseline plus 80%, the alarm set point would be 9 cmH2O. If a minimum over baseline requirement was employed at baseline plus 5 cmH2O and baseline PEEP was 0 cmH2O, the alarm set point would be 5 cmH2O.

In some cases, a low PEEP alarm may be determined using baseline minus a fixed amount. In such cases, the alarm set point may comprise baseline minus 0-20 cmH2O, such as baseline minus 0-10 cmH2O. Alternatively, the low PEEP alarm may be determined using a percentage formula. In such cases, the alarm set point may comprise baseline minus 0-90% or a minimum value of baseline minus 1-10 cmH2O, such as baseline minus 40-90% with a minimum value of baseline minus 1-5 cmH2O. For example, with a baseline PEEP of 5 cmH2O, using baseline minus 5 cmH2O, the alarm set point would be 0 cmH2O, using baseline minus 80%, the alarm set point would be 1 cmH2O. If a minimum over baseline requirement was employed at baseline plus 5 cmH2O and baseline PEEP was 0 cmH2O, the alarm set point would be 5 cmH2O.

Alarm and ventilation parameter settings may be adjusted from these described values at any time and if desired, stored for the specific user, institution, hospital group, patient, patient group, etc.

The user may then be asked to confirm/accept the Baseline Ventilation Parameters 1020 (if not done previously) and the Baseline Alarm Conditions 1040, as part of User Confirmation 1050, or, in closed-loop operation, the Ventilator 500 and/or Database 200 will accept those settings. Thereafter, the user can Initiate Ventilation 1060.

### Execution of Alarm Workflow During Ventilation

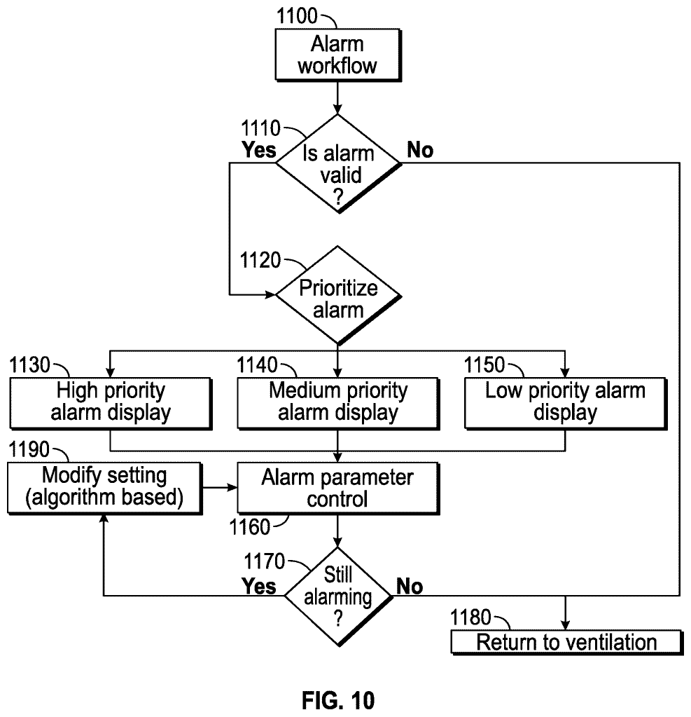

When an alarm is triggered, the Ventilator 500 and/or Database 200 may go into the Alarm Workflow 1100 algorithm while maintaining ventilation, an example of which is shown in FIG. 10. When an alarm is triggered, the Alarm Validity 1110 may be checked to ensure it is a valid alarm. By improving data fidelity though the use of one or more of algorithms, AI/ML, and audio/video analysis, some or all false alarms can be identified as false positives and not be triggered. False positives can be caused by many factors, including but not limited to a poor waveform recording (such as from an EKG), the patient moving and disrupting the signal, or sensor(s) being disconnected (e.g. an O2 saturation sensor falling off the finger of a patient). If the alarm is determined or suspected not to be valid, the Ventilator 500 may Return to Ventilation 1180 and continue the ongoing ventilation workflow algorithm. If the alarm determine or suspected to be valid, the Ventilator 500 and/or Database 200 may Prioritize the Alarm 1120. Alarm priorities may follow, e.g., the ISO 80601 and IEC 60601 requirements, as applicable (e.g., high airway pressure is a high priority alarm, internal electrical power nears depletion a medium priority alarm, and switchover to internal electrical power a low priority alarm). Once the alarm priority is determined, the Ventilator 500 and/or Database 200 may enter Alarm Parameter Control 1160. During Alarm Parameter Control 1160, the Ventilator 500 and/or Database 200 may continually evaluate if the alarm condition is still met 1170. In Alarm Parameter Control 1160, there may be multiple options in Modify Setting 1190. High Priority Alarms 1130 are immediately acted upon, whereas Medium Priority Alarms 1140 and Low Priority Alarms 1150 may be acted upon immediately, have a delayed action, or just provide an indication of the alarm state. There are alarm conditions that can potentially be mitigated by modifying ventilation parameters (e.g., high airway pressure) and those that cannot (e.g., low internal electrical power). Alarm conditions that cannot be mitigated by modifying ventilation parameters may be left in the alarm state until acted upon by the user. Alternatively, alarm conditions that can potentially be mitigated by modifying ventilation parameters may be acted upon by the Ventilator 500 and/or Database 200. This action may comprise presenting recommended changes to the user, directly changing ventilation parameters, or some combination thereof.

Actionable High Priority Alarms 1130 can have the parameter adjusted up or down using one or more of a fixed amount, percentage, variable amount (e.g., using a progressive, interval, derivative (PID) control algorithm), amount as determined by AI/ML as described herein, or some combination thereof. One example of a ramping percentage reduction for a high priority alarm is as follows. In this example, the high priority alarm is triggered by high airway pressure; however, other triggers and parameter adjustments are possible. The VT is set at 500 mL and the high airway pressure alarm is set at 30 cmH2O. The airway pressure is measured during an inspiratory cycle to reach 38 cmH2O. The Ventilator 500 (or Database 200) immediately terminates delivery of any additional gas during that breath. The calculated volume delivered during the shortened breath is, e.g., 400 mL. The Ventilator 500 tries the next breath at the original VT of 500 mL. If that breath continues to trigger the alarm, the next breath is reduced. The calculated volume delivered during the shortened breath is, e.g., 400 mL. In this example, the algorithm takes the original reduced amount (100 mL) and multiplies it by a percentage factor, such as a ramping 40%, 30%, 20%, 10%, such that he next breath will be 500 mL−(100 mL×0.40)=460 mL. If 460 mL no longer triggers the high airway pressure alarm, then 460 mL is delivered for a specified number of breaths, and the VT is then ramped back up to with the original VT or until the high airway pressure alarm is triggered. Ramp back up to the original setting may comprise the same types or combinations of operations as the ramp down. If 460 mL triggers the alarm again, the above-described process continues at 30% of the original reduced amount: 460 mL−(100 mL×0.30)=430 mL. As such, 430 mL is delivered on the next breath. This may then continue with 20% (delivering 410 mL) and then stay at 10% reductions (e.g., 400 mL, 390 mL, 380 mL, etc.) until the high airway pressure alarm is no longer triggered. When ramp up is initiated, the reverse order (or any other parameter adjustment method) may be used. For example, if the high airway pressure alarm stopped triggering at 400 mL, then the ramp up may go 10% to 410 mL, then 20% to 430 mL, then 30% to 460 mL, then 40% to 500 mL. The VT may stay at the original VT of 500 mL until any further action was input or deemed necessary (e.g., by an alarm being triggered).

In another example, the alarm workflow may be adapted for a medium priority alarm. Although the medium priority alarm will be described as having met the high PEEP alarm conditions, with a concern of having auto-PEEP, and using a fixed amount parameter adjustment, other alarm conditions and parameter adjustments are possible. In this example, the original ventilation settings comprise PEEP at 10 cmH2O, VT at 500 mL, inspiratory flow rate at 60 L/min, I:E ratio at 1:2, and respiration rate at 10 bpm. The high PEEP alarm is set at 12 cmH2O. The high PEEP alarm conditions are met with a PEEP of 15 cmH2O. As this is a medium priority alarm and allowable for high PEEP, the Ventilator 500 waits for three consecutive breaths that meet the high PEEP alarm conditions. If after 3 consecutive breaths the high PEEP alarm conditions are no longer met, the Ventilator 500 does not trigger the high PEEP alarm. If the high PEEP alarm conditions are still met, then the following parameters may be sequentially or in any combination adjusted to increase the PEEP. The parameters include, but are not limited to, reducing the tidal volume, extending the expiration time, increasing the inspiratory flow rate, increasing the I:E ratio, and/or decreasing the respiration rate. The Ventilator 500 and/or Database 200 may also rank order the parameters as to which parameter(s) gets changed first, such as 1) increase inspiratory flow rate, 2) increase I:E ratio, 3) decrease respiration rate, then 4) decrease VT. In this example, the inspiratory flow rate is increased by 10 L/min for three consecutive breaths to 70 L/min. If the high PEEP alarm conditions are still met, then for the next three breaths the inspiratory flow rate is increased to 80 L/min. This continues until either the alarm conditions are no longer met or the maximum for any parameter is reached. In this example, the maximum flow rate is set at 100 L/min. When 100 L/min is reached, and if the high PEEP alarm conditions are still met, then the Ventilator 500 and/or Database 200 start adjusting the next parameter, such as decreasing the respiration rate or decreasing VT.

Multiple parameters may be adjusted before any one parameter reaches its maximum or minimum value. Continuing the example above, the flow rate reaches 80 L/min and is still meeting the high PEEP alarm conditions. On the next series of breaths, the flow rate may be increased by another 10 L/min, and, concurrently, the VT may be decreased following any of the previously described parameter adjustment operations. One or more parameters can be adjusted to return the original alarming parameter back to baseline (e.g., using previously described operations). Any time the ventilation parameters are changed, the Ventilator 500 displays the updated information and the Database 200 is updated as previously described.

### Maintaining Ventilation

After initiating ventilation, the Ventilator 500 and/or Database 200 may prompt a request for new ABG data. This request may occur, e.g., 5-30 minutes after initiating ventilation, such as 5-15 minutes after initiating ventilation. The ABG data can be manually entered, scanned, or wirelessly or otherwise accessed (e.g., from an externa database) and input to the Ventilator 500 and Database 200. This ABG data is an example of many possible data Inputs 1070 (e.g., heart rate, blood pressure, temperature, EKG, Plateau Pressure Maneuver 1075 data, alarm information, and settings, etc.). The Ventilator 500 and/or Database 200 maya evaluate the ABG data for a number of parameters and conditions to determine if they are in a normal range or out of range. The parameters/conditions and their normal ranges may include, but are not limited to, hypoxemia, PaO2 (80 to 100 mm Hg), SaO2, 95% to 100%, pH, 7.35 to 7.45, PaCO2, 35 to 45 mm Hg, and HCO3-, 22 to 26 mEq/L.

In addition to prompting for ABG data, the Ventilator 500 and/or Database 200 may prompt for or automatically conduct a Plateau Pressure Maneuver 1075. With user confirmation, or automatically, the Ventilator 500 may perform an expiratory hold maneuver for, e.g., 0.5-2.0 seconds, more preferably 0.5-1.0 seconds. From the Plateau Pressure Maneuver 1075, the Ventilator 500 and/or Database 200 may determine and optionally display the peak airway pressure, plateau pressure, and the difference between the peak airway pressure and the plateau pressure. Optionally, the Ventilator 500 and/or Database 200 may calculate the plateau pressure passively from the expiratory flow curve. The Ventilator 500 and/or Database 200 may calculate and display parameters including, but not limited to, one or more of static compliance (mL/cmH2O)=tidal volume/(Pplat−PEEP) or dynamic compliance from the waveform; resistance: airway resistance (Raw)=(PIP−Pplat)/Flow; and Auto-PEEP: Auto-PEEP=(total PEEP−set PEEP). The Ventilator 500 and/or Database 200 may evaluate each individual parameter, as well as subsets of parameters or all parameters, and may apply calculations to determine the change and timing of change of each or of groups of parameters to obtain the desired effect. The Ventilator 500 and/or Database 200 can use PID type of control for settings change, AI/ML, other algorithms, etc. to make changes or remain at the same settings.

Once new Inputs 1070 have been received by the Ventilator 500 and/or Database 200, the user may be prompted to confirm/implement the new settings, or the new settings are automatically implemented depending on the mode of operation, New settings for both ventilation parameters and alarm settings based on calculations, underlying conditions, current values in one or more parameters, trends in one or more parameters, or any combination thereof may be determined and acted upon as part of the Update Ventilation Parameters and Alarm Conditions 1080 workflow step. In example of updating the ventilation parameters, after the Plateau Pressure Maneuver 1075 is conducted, the Ventilator 500 and/or Database 200 determines that the plateau pressure is high with a target of <30 cmH2O (determined by the guidelines for that type of patient stored in or accessed by the Ventilator 500 and/or Database 200) and an actual value of 36 cmH2O. The Ventilator 500 and/or Database 200 prompts or directly changes the VT to a lower amount using PID type of control, AI/ML, or other algorithms described herein until the plateau pressure is <30 cmH2O. All other parameters may be checked to ensure they remain within the desired ranges. The alarm conditions may then be updated to account for the lower setpoint for VT, as previously discussed above with respect to setting Baseline Alarm Conditions 1040. The Ventilator 500 and/or Database 200 may prompt for input or (if connected/controlled) automatically receive ABG data and other Inputs 1070. This data can be prompted/received at determined, varying timepoints or upon a certain change or trend patient parameters/conditions. For example, the ABG or other data may initially be prompted/received at a fixed interval of 30-60 minutes. Other timings may be specified by a user. Alternatively, the Ventilator 500 default settings can be used. If the patient is stable and the ventilation is unchanged for a given duration (e.g., 2× the original interval time, or 2-5 data input cycles for that parameter), then the duration between prompted/received is extended (e.g., current duration +25-100% increase in the time before the next reading). If the patient is unstable, the interval may be maintained; reduced, e.g., by 10% to 75%; or reduced down to a minimum time, which may be user selectable or defaulted to, for example, 15 minutes or less. In some cases, the time may be more than 15 minutes.

Ventilation of the patient may continue with the Ventilator 500 and/or Database 200 evaluating any Inputs 1070, alarms, and changing patient conditions to determine any appropriate changes to the ventilation and/or alarm settings. The Ventilator 500 and/or Database 200 may continuously or discretely assess a patient's readiness to be weaned from mechanical ventilatory support: Ready to Wean 1085. The Ready to Wean 1085 criteria are entered into, stored in, or accessed by the Ventilator 500 and/or Database 200 based on recommendations/best practice, such as hospital, local, regional, national, or global guidelines, or requested from the user, and can include but are not limited to respiratory rate, VT, minute ventilation, PEEP, pH, PaO2/FiO2, Shunt (Qs/Qt), negative inspiratory force, frequency/VT, stable cardiovascular status, heart rate, hemoglobin, systolic blood pressure, temperature, and no or minimal vasopressor or inotrope. Example, non-limiting values for these parameters in an adult are respiratory rate less than 35 bpm, VT greater than 5 mL/kg, vital capacity greater than 10 mL/k, minute ventilation less than 10 L/min, PaO2>60 and PCO2≤60 mmHg, PEEP ≤8 cmH2O, pH ≥7.30, PaO₂/FIO₂ greater than 200 or SaO₂>90% on FiO2≤0.4, shunt (Qs/Qt) less than 20%, negative inspiratory force less than (more negative) −25 cmH2O, frequency/Vt less than 105, or less than 130 in elderly patients, heart rate <140 beat/minute, hemoglobin ≥8 g/dL, systolic blood pressure 90-160 mmHg, temperature 36-38° C., and no or minimal vasopressor or inotrope (<5 μg/kg/minute dopamine or dobutamine). In addition, Inputs 1070 for subjective measures may prompted by the Ventilator 500 and/or Database 200 requiring user input and/or through the use of Audio/Visual Equipment 1400 and image processing.

### Weaning

Once the patient has met the Ready to Wean 1085 criteria, the Ventilator 500 may prompt for or automatically conduct a spontaneous breathing trial (SBT) or other similar weaning trial as part of the Wean Workflow 1200. In an example, the weaning trial comprise an SBT. The Ventilator 500 and/or Database 200 proves the baseline setting(s) based on patient data, which have been previously obtained, and any other relevant Inputs 1070. The user may be prompted to adjust any parameters prior to starting the SBT. The duration of the SBT is set at, e.g., 30-120 minutes. The patient is placed in pressure support ventilation mode with low level support; pressure support and PEEP at 0-10 cmH2O, such 5-8 cmH2O or 5-7 cmH2O and 5 cmH2O, respectively. The Ventilator 500 runs the SBT for 30 minutes unless failing criteria are met. If failing criteria are met, the Ventilator 500 immediately resorts to ventilating the patient at the pre-SBT settings and the user is alerted that the patient failed the SBT. After 30 minutes, the user is prompted to review the patient's parameters and the decision to continue the SBT up to 120 minutes is determined, or this is done automatically by the Ventilator 500 and/or Database 200. Primary SBT failing parameters may comprise respiratory rate not within limits (<8 bpm or >35 bpm), SpO2<88%, delirium, or respiratory distress. Once the patient passes the SBT and it is determined Wean Successful 1210, the user may be alerted of the result and may be presented or access all the data related to the SBT. The user can then Remove Patient from Ventilation 1220.

If the patient fails the SBT, the Ventilator 500 and/or Database 200 evaluate the data and can present the user with an estimation of the reason for the SBT failure. These include but are not limited to hypoxemia (V/Q mismatch), diaphragmatic fatigue (elevated RSBI and/or development of acute respiratory acidosis), restrictive physiology, volume overload, etc.

Weaning trials other than SBT are contemplated herein and may be comprised in methods and systems described herein.

### Examples

An example of an O2U System 100 embodiment is a Control Center 300 operating in closed loop with controlled equipment, more specifically, a Patient Monitor, Ventilator 500, and multiple IV Pumps 1500. The Control Center 300 is monitoring every breath, with the Database 200 calculating all ventilation parameters and comparing to medications and patient data (EKG, temp, pressures, SpO2, etc). The Database 200 using AI/ML determines that the patient's physiologic parameters are such that the patient is over sedated. The Database 200 calculates all the necessary changes and sends that information to the Display 250. The Display 250 provides the user the opportunity to make or edit the changes, if no action is taken by the user in a set amount of time (e.g., 1 minute), the Database 200 commands a first IV Pump 1500 delivering anesthetic to decrease the delivery rate by the calculated amount, commands a second IV Pump 1500 delivering saline to increase the delivery of saline, and commands the Ventilator 500 to increase minute volume by the calculated amount. These changes were made to decrease intake of anesthetic, offload anesthetic already in the patient's system, and balance physiologic parameters. The Database 200 updates all of the patient's and equipment parameters, trends, and treatment history as well as determines the new patient treatment trajectory and makes all of this available for display on the Display 250, Ventilator UI 510, Handheld Communication Device 1300, if capable, the IV Pump 1500 UI, and other capable components.

In another example, a patient is a 60-year-old male, Hispanic, 185 cm height, 80 kg, fever (101.3° F.), with asthma, and is admitted for labored breathing. The Database 200 receives all the relevant and/or available data. The user request the diagnostic/treatment scenario(s) based on specified criteria. The criteria may be broad, such as adult male+asthma, or the criteria may be more specific, such as 60-65 years old, male, Hispanic, 180-190 cm height, 70-80 kg, with asthma. Database 200 evaluates all the patient records available based on the provided criteria and provides a display of the patient's condition, likely diagnosis, proposed diagnostics, therapy/treatment, and bounding parameters/statistics for this information. The user may then increase or decrease the criteria and see the resulting effects on this information. If the statistics are insignificant and/or the sample size determined to be too small, the user may adjust any parameters to increase the sample size, such as by increasing the age range, increasing the weight range, removing the race characteristic, or adjusting any other criteria discussed herein. Conversely, the sample size may be reduced by adding additional and/or stricter criteria (e.g., smaller age, weight, and height ranges, etc.). Once the user is satisfied with the criteria and statistics, the Database 200 may provide the patient's condition, likely diagnosis, proposed diagnostics, therapy/treatment, and bounding parameters/statistics for these criteria. In this example, the Database 200 may display that 92% (667 of 725) of similar patients had bronchospasm, should receive a bronchodilator (type and dose regimen), CPAP ventilation of 100% FiO2 at 5 cmH2O for 2-4 hours until the SpO2 is >98%, then be weaned off the O2 over a duration of 2 hours, and if the SpO2 remains >98%, for an additional 1 hour. At this points, the similar patients would then have completed the therapy for the immediate admission and be recommended for a 1-5 day post therapy evaluation. Potential concerns may be inability to sufficiently dilate airways requiring mask ventilation (and provide the parameters if requested), underlying worsening respiratory condition (e.g. COPD, of which 4% of patients were diagnosed with (e.g. 29 of 725)), and other outcomes.

Using rapid iterative processing with intelligent algorithms may be extremely valuable in dynamic situations, such as where the patient's conditions are quickly changing, the treatment condition is rapidly changing, and/or the disease is evolving or just becoming understood. An example would be during the COVID-19 outbreak, ventilation strategies were varied throughout treatment centers with some working well and others not so well. The Database 200, using a suite of AI/ML and related computational algorithms, such as those described herein, may enable evaluating a patient's data and therapy and providing the user with the latest treatment regimens that have obtained the most success. Ventilation parameters and strategies can evolve quickly. As such, the Database 200 may enable best practices to be determined based on all information and wirelessly updates the Control Center Display 250, Ventilator UI 510, and/or Handheld Communication Device 1300 display, thereby reducing the time for the best practices to be determined, the users to be informed and updated, and the controlled equipment software, operating parameters, and displays to be upgraded to provide the best treatment options. In addition, this standardizes the treatment which in turn more rapidly increases the data available, allowing the improvement of machine learning predictions.

In another embodiment, by collecting and curating extensive amounts of ventilator waveform data in the Database 200, this information may be used as test, training, and validation data for the construction and deployment of machine learning classification algorithms such as those described above. These algorithms may include a combination of supervised and unsupervised predictors which take as input labeled or unlabeled ventilator data to predict given patient outcomes, including but not limited to ventilator dyssynchrony, breath stacking, or auto-peep detection. This information may then be combined with other related patient data, such as disease specific information; demographic data; ABG; blood pressure; body temperature; heart rate; respiration; general health; patient position; respiratory rate; VT; minute ventilation; PEEP; pH; PaO2/FiO2; pulmonary shunt fraction; negative inspiratory forces; frequency; stable cardiovascular status; heart rate; hemoglobin; systolic blood pressure; vasopressor or inotrope; trigger sensitivity; flow rate; waveform; and inspiratory/expiratory (I/E) ratio; images; audio; and videos, to give a holistic view of potential patient outcomes. Once the Database 200 contains ample amounts of ventilation and patient data, further deep learning (DL) algorithms, such as those described herein above, may be constructed to answer more complex questions, including but not limited to, higher order correlation analyses of different ventilator outcomes and their interaction and relationship with any particular disease. These DL methods are powerful tools that can elucidate and predict extremely complex higher order relationships between given features that more simple ML algorithms are unable to do.

AI/ML and/or audio/visual analysis may also be used to improve data fidelity. In one embodiment, the AI/ML looks at past and current data/parameters from one or more sources and determines if the condition is accurate/valid or not. The O2U System 100 may also activate or send commands to other pieces of controlled equipment or request user input to modify parameters, take measurements, or send data to gain additional information in ascertaining if the condition is accurate. If the condition is accurate, the Database 200 can affect any necessary next steps, such as triggering and alarm, modifying equipment operating parameters, etc. If the condition is not accurate, the Database 200 can inform the user of the issue, trigger a different alarm or alert, and/or provide information on how to rectify the issue. In an example, the patient's EKG signal drops out, or the signal shows a loss of a QRS peak and the EKG does not recognize this as a contraction. The immediately prior EKG data is evaluated along with the prior and current blood pressure/heart rate data (not taken from the EKG), as well as patient visualization. A blood pressure cuff can be activated by the Database 200 or other controlled equipment (or the user) to take the patient's blood pressure and heart rate, and if there is a normal heart rate (compared to immediately prior to the loss of EKG heart rate signal), and visualization shows no abnormal changes to the patient, the Database 200 can determine that the patient's heart is still beating and circulating blood, not trigger the heart rate alarm, and inform the user by placing an alert on the Display 250, Handheld Communication Device 1300, or other controlled equipment that there is an issue with the EKG reading, but not with the patient's heart rhythm.

In another embodiment, the O2U System 100 may measure the patient's SpO2, as measured by a sensor placed on the patient, such as on their finger (or other extremity). If the SpO2 value takes on a significant change over a given time period (e.g., >5% change over 5 seconds), AI/ML data recognition may determine if the SpO2 sensor is properly attached to the patient. The Database 200 may also query other equipment readings to determine the validity of the condition and whether to trigger an alarm (accurate/valid condition) or inform the user of the issue, trigger a different alarm or alert, and/or provide information on how to rectify the issue with the sensor reading.

In another embodiment, the Audio/Visual Equipment 1400 and other data inputs can be used to improve ventilation of the patient. The Audio/Visual Equipment 1400 data related to patient position and patient comfort may be correlated to the ventilation parameters from the Ventilator 500 and other Inputs 1070 (e.g. ABG data, heart rate, blood pressure) by the Database 200, to determine the optimum position to achieve the target ventilation goals, with the physiologic parameters as desired levels, as well as a minimized amount of patient effort to achieve these goals and prevent ventilation dyssynchrony. In addition, as the patient cannot always be in one position, the Database 200 can also determine the optimum ventilation parameters and other therapy settings (e.g., anesthesia level) for each position the patient is or may be in. The Database 200 can inform the user of when and why they should move the patient and to what position to achieve ventilation goals, as well as reduce injury to the patient from being in a single position for too long of a duration.

The greater the amount of data from various inputs, the greater the opportunity to evaluate the information to determine accuracy of any reported patient parameter. Having controlled equipment and/or other data inputs evaluated and cross-checked by the Database 200 enables improved data fidelity and reporting compared to the current standard of individual pieces of equipment only evaluating their own measured data.

In another embodiment, the Audio/visual Equipment 1400 data may comprise image data. Correlating this image data with other patient parameters allows the construction of more holistic ML models that can yield better classification results than models that only take in numerical data. This imaging data can also be used to construct deep learning models that can accurately predict given patient outcomes including, but not limited to, discomfort, cough, survival, prognosis, other endpoints, response to treatment, and other combinations thereof.

It is also contemplated that in some embodiments the software and systems described herein comprise devices and/or controlled equipment other than a ventilator. By way of nonlimiting example, such equipment may comprise general medical equipment such as cardiac monitors, IV Pump 1500, or blood gas analyzers; medical imaging equipment such as a fluoroscope, X-ray machine, ultrasound probe, intravascular ultrasound equipment; EKG or EEG equipment; or other equipment such as intermittent pneumatic compression (IPC), patient bed and bed settings (e.g. position), thermal (hot/cold), and pads/blankets. In some embodiments, the controlled equipment may further collect, process, or send information to the Database 200 or Control Center 300. In some embodiments, the information sent to the Database 200 may be used as test, training, and validation data to inform AI/ML models. In some embodiments, the controlled equipment uses (an) algorithm-driven workflow(s) and/or include the use of AI/ML either internally or accessed through the Database 200 to provide for open, semi-open, or closed loop operation.

It is also contemplated that in some embodiments the software and systems described herein include more than one device or piece of controlled equipment. The more than one device or pieces of controlled equipment may comprise any device or piece of equipment disclosed above as well as other devices or pieces of equipment. The more than one device or pieces of controlled equipment may collect, store, process or send information to the Database 200 or Control Center 300. In some embodiments, the information may be used in (an) algorithm-based workflow(s) or AI/ML internally in the more than one piece of controlled equipment or accessed through the Database 200 to provide for open, semi-open, or closed loop operation of an entire patient therapy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system, the system comprising:
(a) medical equipment;
(b) a processor; and
(c) a non-transitory computer-readable medium including software comprising artificial intelligence, the software configured to cause the processor to:
(i) receive an input comprising a quantifiable parameter from the medical equipment;
(ii) analyze the input using the artificial intelligence; and
(iii) modify performance of the medical equipment resulting in a quantifiable improvement in the quantifiable parameter.

2. The system of claim 1, wherein the medical equipment comprises a ventilator.

3. The system of claim 2, wherein the quantifiable parameter comprises ventilator data.

4. The system of claim 1, wherein the medical equipment comprises at least one of: a cardiac monitor; an intravenous pump; a blood gas analyzer; medical imaging equipment; an electrocardiogram; intermittent pneumatic compression equipment; bed settings; and thermal pads and blankets.

5. The system of claim 4, wherein the quantifiable parameter comprises data from the medical equipment.

6. A system, the system comprising:
(a) medical equipment;
(b) a processor; and
(c) a non-transitory computer-readable medium including software comprising artificial intelligence, the software configured to cause the processor to:
(i) receive, at a remote location, an input comprising a quantifiable parameter from a patient receiving support from the medical equipment;
(ii) analyze, at the remote location, the input using the artificial intelligence; and
(iii) send instructions, from the remote location, to modify performance of the medical equipment resulting in a quantifiable improvement in the quantifiable parameter.

7. The system of claim 6, wherein the medical equipment comprises a ventilator.

8. The system of claim 6, wherein the remote location controls the medical equipment.

9. The system of claim 8, wherein the medical equipment provides audio or visual capabilities at the remote location.

10. The system of claim 9, further comprising a display at the remote location, the display showing the quantifiable parameter captured by the medical equipment.

11. A system for providing support to a patient, the system comprising:
(a) medical equipment;
(b) a processor; and
(c) a non-transitory computer-readable medium including software comprising artificial intelligence, the software configured to cause the processor to:
(i) receive an input comprising a quantifiable parameter from a patient receiving support from the medical equipment;
(ii) model the input using the artificial intelligence; and
(iii) modify performance of the medical equipment using the model to result in optimized performance of the medical equipment.

12. The system of claim 11, wherein the medical equipment comprises a ventilator.

13. The system of claim 12, wherein the model optimizes performance of the ventilator.

14. The system of claim 11, wherein the optimized performance is achieved by comparing the input to the model.

15. The system of claim 11, wherein the software is further configured to cause the processor to train the model with a data set of parameters from patients, wherein the model is a machine learning model.

16. A system for providing support to a patient, the system comprising:
(a) medical equipment;
(b) a processor; and
(c) a non-transitory computer-readable medium including software comprising artificial intelligence, the software configured to cause the processor to:
(i) receive an input comprising a quantifiable parameter from a patient receiving support from the medical equipment;
(ii) analyze the input using the artificial intelligence; and
(iii) modify performance of the medical equipment using a defined mode of operation that results in a quantifiable improvement in the quantifiable parameter.

17. The system of claim 16, wherein the medical equipment comprises a ventilator.

18. The system of claim 16, wherein the defined mode of operation is an open loop operation, wherein all changes to the performance of the medical equipment are suggested by the artificial intelligence.

19. The system of claim 16, wherein the defined mode of operation is a semi-open loop operation, wherein some changes to the performance of the medical equipment are suggested by the artificial intelligence, and wherein other changes to the performance of the medical equipment are made by the artificial intelligence.

20. The system of claim 16, wherein the defined mode of operation is a closed loop operation, wherein all changes to the performance of the medical equipment are made by the artificial intelligence.
